(19) United States
(12) Patent Application Publication
(10) Pub. No.: US 2014/0249056 A1
(43) Pub. Date: Sep. 4, 2014

(54) BILAYERS

(71) Applicant: ISIS INNOVATION LIMITED, Oxford (GB)

(72) Inventors: Mark Ian Wallace, Oxford (GB); Sebastian Leptihn, Tuebingen (DE); James Russell Thompson, Cheshire (GB)

(73) Assignee: ISIS INNOVATION LIMITED, Oxford (GB)

(21) Appl. No.: 14/174,634

(22) Filed: Feb. 6, 2014

Related U.S. Application Data
(63) Continuation of application No. 13/389,197, filed on May 15, 2012, now abandoned.

(30) Foreign Application Priority Data
Aug. 7, 2009 (GB) 0913823.1

(51) Int. Cl. G01N 33/543; G01N 33/68

(52) U.S. Cl. CPC G01N 33/543; G01N 33/6842
USPC 506/32

(57) ABSTRACT

A method for producing a bilayer, the method comprising: (a) providing a hydrated support and a hydrophilic body immersed in a hydrophobic medium; wherein a first monolayer of amphipathic molecules is formed on an interface between the hydrophobic medium and the hydrophilic body and a second monolayer of amphipathic molecules is formed on an interface between the hydrophobic medium and the hydrated support; and (b) bringing the first monolayer into contact with the second monolayer to form a bilayer of amphipathic molecules, wherein at least part of a cell membrane, comprising cell membrane constituents, is provided in or on the hydrated support and/or in the hydrophilic body, and such that constituents of the cell membrane incorporate into the bilayer during or after the bilayer formation. A bilayer produced by the method of the invention, and uses of the bilayer.

The present invention relates to a bilayer, such as a lipid bilayer, to a method of producing a bilayer, and to the use of a bilayer.

Artificial planar lipid bilayers serve as simplified models of biological membranes and are widely used for the electrical characterisation of ion-channels and protein pores (Mueller, P. et al., 1962. Nature 194, 979-980; White, S. H. 1986. ed. Miller, C. Plenum Press: New York).

Patch-clamping is also used to study biological membranes, for example, patch clamping is often used in single channel recording (SCR) (Sakmann, B. & Neher, E. 1984. Annual Review of Physiology 46, 455-472). Other methods used include excised-patch, tip-dip and on-chip methods.

Patch-clamping of whole cells is a versatile and sensitive means of examining channels, but is very time-consuming and difficult, and often complicated by the heterogeneous nature of cell membranes. In contrast, artificial planar lipid bilayers control the constituents of the system and can be used to study purified proteins.

Planar lipid bilayers are usually formed either by painting, where a solution of lipid in an organic solvent is directly applied to an aperture separating two aqueous compartments (Mueller, P. et al., 1962. Nature 194, 979-980; White, S. H. 1986. ed. Miller, C. Plenum Press: New York), or variants of the Langmuir-Blodgett technique, where two air/water monolayers are raised past an aperture (Montal, M. & Mueller, P. 1972. Proceedings of the National Academy of Sciences of the United States of America 69, 3561-3566). Although widely used, planar lipid bilayers are difficult to prepare, and their short lifetime prohibits their use in many situations.

Alternative emulsion-based approaches to forming bilayers have also been proposed (Tsofina, L. M. et al., 1966. Nature 212, 681-683), where bilayers are created between aqueous surfaces immersed in a solution of lipid in oil. When immersed in an immiscible lipid/oil solution, aqueous surfaces spontaneously self-assemble a lipid monolayer (Cevc, G. 1993. Phospholipids handbook, ed. Cevc, G., Marcel Dekker, New York); Seddon, J. M. & Templer, R. H. 1995. eds. Lipowsky, R. & Sackmann, E., Elsevier, Amsterdam, Oxford), and when monolayers from two aqueous components are brought into contact they can ‘zip’ together to form a lipid bilayer (Tien, H. T. 1974. M. Dekker, New York; Fujiwara, H. et al., 2003. Journal of Chemical Physics 119, 6768-6775). Recent studies have shown that microfluidic flows (Malmstadt, N. et al., 2006. Nano Letters 6, 1961-1965; Funakoshi, K. et al., 2006. Analytical Chemistry 78, 8169-8174) and droplets (Funakoshi, K. et al., 2006. Analytical Chemistry 78, 8169-8174; Holden, M. A. et al., 2007. Journal of the American Chemical Society p 8650-5) can be contacted in a lipid/oil solution to create bilayers suitable, for example, in single-channel recording experiments.

More recently, a previous patent application, namely publication number WO2009024775 (A1), describes the formation of an artificial lipid bilayer by immersion of a hydrated support (e.g. a hydrogel) and a hydrophilic body (e.g. an aqueous droplet) in a lipid-in-oil solution resulting in the formation of a stable artificial bilayer between the hydrated support and hydrophilic body. Such a bilayer may be used for study of detergent solubilised proteins inserted into the bilayer. However, this limits the variety of proteins that can be successfully studied.

It is an object of the invention to improve the analysis and study of membrane constituents in bilayers.

According to a first aspect of the invention, there is provided a method for producing a bilayer, the method comprising:

(a) providing a hydrated support and a hydrophilic body immersed in a hydrophobic medium;
wherein a first monolayer of amphipathic molecules is formed on an interface between the hydrophobic medium and the hydrophilic body and a second monolayer of amphipathic molecules is formed on an interface between the hydrophobic medium and the hydrated support; and
   (b) bringing the first monolayer into contact with the second monolayer to form a bilayer of amphipathic molecules,
wherein at least part of a cell membrane, comprising cell membrane constituents, is provided in and/or on the hydrated support and/or in the hydrophilic body, and
such that constituents of the cell membrane incorporate into the bilayer during or after the bilayer formation.

Surprisingly, the incorporation of the cell membrane with the aqueous components (hydrated support and/or hydrophobic body) leads to the obtained bilayer incorporating at least some of the constituents of the cell membrane. Therefore any membrane, or membrane protein of interest can be easily reconstituted in a bilayer from fragments of cell membranes or from entire intact cell membranes, or from purified eukaryotic membrane constituents. This enables straightforward in vitro characterisation, e.g. by electrical recording or fluorescence microscopy, of such a membrane protein. This is in contrast to prior art techniques that generally involve the incorporation of purified membrane proteins or proteoliposomes to an existing bilayer rather than directly from cell membranes.

Accordingly, the method of the invention not only spontaneously forms a bilayer of amphipathic molecules but also spontaneously incorporates the cell membrane constituents from the hydrated support and/or the hydrophilic body.

The present invention provides an advantage that membrane constituents are readily incorporated into a bilayer for study and minimal sample preparation is required. For example, any membrane protein can be reconstituted into a bilayer using the method of the invention. In particular, the technique allows for eukaryotic membrane proteins derived from membrane preparations to be reconstituted into bilayers.

To date, eukaryotic cell membrane constituents have been incorporated into artificial bilayers. However, these experiments require lengthy detergent-based purifications of the target protein and reconstitution into proteoliposomes, and/or the fusion of proteoliposomes with a bilayer. Each of these steps is difficult. It is therefore particularly beneficial that the method permits such eukaryotic membranes to be easily incorporated into artificial bilayers. The straightforward reconstitution of eukaryotic membrane proteins and other membrane constituents enables in vitro studies of eukaryotic membrane protein function as well as studies of membrane constituents other than proteins, previously impossible with the prior art.

The ease of use provides considerably greater potential for high throughput than conventional methods for studying cell membrane proteins and other membrane constituents.

Another benefit of the method is that very little cellular material is required, e.g. less than one cell may be required per bilayer, due to highly efficient reconstitution. Membrane proteins having low levels of expression may be used, circumventing the need for well-developed cell lines to stably express membrane proteins of interest (for example, as in whole-cell patch clamping). Additionally, live cells are not required, enabling the long-term storage of membrane protein samples (membrane material) for future use.

The invention provides a benefit of straightforward single-molecule measurements or bulk measurements of natural cell membrane constituents, allowing mechanistic insights into binding of molecules of interest.

Advantageously, there is no need to prepare cell membrane constituents for study in a surfactant or detergent other than their native lipid environment. For example, there is no requirement for prior preparation of defined proteoliposomes (protein-containing lipid vesicles) for membrane protein reconstitution.

Advantageously, the invention allows the study of cell membrane constituents, such as membrane proteins, where the protein's functional expression, post-translational modification in the eukaryotic expression system and lack of requirement for detergent purification allows for the maintenance of protein functionality.

Reconstitution of the membrane proteins of interest into bilayers appears to result in no alteration in activity. Cytosolic protein domains of membrane proteins remain in an aqueous environment through the reconstitution procedures highlighted.

In addition, the protein is not removed from its native lipid-embedded environment, in contrast to detergent purification protocols, where native behaviour might be lost due to protein denaturation of either hydrophobic membrane-spanning domains or hydrophilic domains.

Direct reconstitution of eukaryotic membranes or membrane fragments, containing membrane constituents of interest such as membrane proteins, avoids the often perceived notion that heterologous protein expression in bacteria is a necessity for sufficient quantities of protein for transfer from expressing system to an artificial lipid bilayer. Expression in a eukaryotic cell line for example allows for post-translational modifications to be performed, which have been shown to be a requirement for correct folding, oligomerisation and function of eukaryotic membrane proteins.

The terms “contacting” or “contact” used herein with reference to the contacting of monolayers of amphipathic molecules to form a bilayer are understood to mean actual physical contact, and/or close enough proximity, to allow the assembly of an amphipathic molecule bilayer from separate amphipathic molecule monolayers.

Where reference is made to “immersed” or “immersing”, the immersion may be partial or complete immersion.

Where reference is made to “membrane protein”, the term includes transmembrane-, integral membrane- and peripheral membrane-proteins, as well as membrane associated or membrane anchored proteins. The membrane protein may be from any organism and from any membrane.

Cell membrane constituents may comprise any components, such as lipids, peptides, proteins and sugars typically found on or in a cell membrane, including internal membranes, such as sub-cellular organelles, and viral membranes.

“Purified membrane constituents” may include membrane associated proteins that have been purified using detergent or artificial lipids.

In step (a) a hydrated support and a hydrophilic body, immersed in a hydrophobic medium, are to be provided, wherein a first monolayer of amphipathic molecules is formed on an interface between the hydrophobic medium and the hydrophilic body and a second monolayer of amphipathic molecules is formed on an interface between the hydrophobic medium and the hydrated support.

It may be that a first monolayer of amphipathic molecules is formed on an interface between the hydrophobic medium and the hydrophilic body due to the provision of amphipathic molecules on the hydrophilic body and/or in the hydrophobic medium.

It may be that a second monolayer of amphipathic molecules is formed on an interface between the hydrophobic medium and the hydrated support due to the provision of amphipathic molecules on the hydrated support and/or in the hydrophobic medium.

In one embodiment, amphipathic molecules are provided in the hydrophobic medium.

The first and second monolayers of amphipathic molecules may consequently self assemble on the hydrated support and the hydrophilic body when each is placed in the hydrophobic medium containing amphipathic molecules.

In another embodiment, amphipathic molecules are provided on the hydrophilic body and/or on the hydrated support.

The first and second monolayers of amphipathic molecules may consequently self assemble from the hydrated support and hydrophilic body when each is placed in the hydrophobic medium.

The provision of amphipathic molecules may be before or after immersion of the hydrated support and/or the hydrophilic body in the hydrophobic medium.

For example, the hydrated support and the hydrophilic body may be added to the hydrophobic medium before or after amphipathic molecules are added to the hydrophobic medium. In a preferred embodiment, amphipathic molecules are added to the hydrophobic medium before the hydrated support and the hydrophilic body are immersed in the hydrophobic medium.

The hydrated support and the hydrophilic body may be immersed in the hydrophobic medium in any order. In a preferred embodiment, the hydrated support is immersed in the hydrophobic medium before the hydrophilic body is immersed in the hydrophobic medium. In another embodiment, the hydrated support is immersed in the hydrophobic medium at the same time as the hydrophilic body is immersed in the hydrophobic medium. In one embodiment, the hydrated support is immersed in the hydrophobic medium after the hydrophilic body is immersed in the hydrophobic medium.

It may be that the cell membrane comprising cell membrane constituents is provided in the hydrated support and/or provided in the hydrophilic body after the bilayer has formed. Alternatively or additionally, the cell membrane comprising cell membrane constituents may be provided in or on the hydrated support and/or in the hydrophilic body before forming the bilayer. Thus the bilayer may be formed first and then brought into contact with the cell membrane. Alternatively, the cell membrane comprising cell membrane constituents may be incorporated at the same time as the formation of the bilayer.

Cell membrane may be provided by layering the cell membrane on top of the hydrated support prior to addition of hydrophobic medium. Cell membrane may, as another option or in addition, be incorporated into the hydrophilic body when the body is formed, or may be injected into the hydrophilic body after it is formed. Cell membrane may, as another option or in addition, be incorporated into the hydrated support when the support is formed, or may be injected into the hydrated support after it is formed. For example, in an embodiment where the hydrated support comprises a hydrogel, the cell membrane may be mixed into the hydrogel prior to setting, or it may be layered on top of the hydrogel after setting. In an embodiment where the hydrated support is glass, or similar solid substrate, the cell membrane may be placed on the top surface of the glass or similar solid substrate.

All reference herein to a cell membrane refers to a cell membrane comprising cell membrane constituents.

In one embodiment, step (a) involves:
providing a hydrated support;
providing cell membrane, by either layering cell membrane on top of the hydrated support, or alternatively by providing the hydrated support in a form where cell membrane is mixed in the hydrated support; and then
immersing the support in a hydrophobic medium that contains amphipathic molecules, resulting in a monolayer of amphipathic molecules being formed on an interface between the hydrophobic medium and the hydrated support; and then
immersing a hydrophilic body in the hydrophobic medium, resulting in a monolayer of amphipathic molecules being formed on an interface between the hydrophobic medium and the hydrophilic body.

In this embodiment, the hydrated support preferably comprises a hydrogel such as agarose. The hydrophilic body is preferably an aqueous droplet, which may be pipetted into the hydrophobic medium. The hydrophobic medium is preferably an oil. The amphipathic molecules are preferably lipids, provided in the hydrophobic medium as a lipid in oil solution. The cell membrane is preferably cell membrane fragments or intact cells provided in aqueous solution which may be mixed with the hydrogel or pipetted onto the hydrated support.

The cell membrane constituents may be provided as a whole cell membrane or as a fragment of a cell membrane, or as a subcellular membrane preparation, or as a subcellular organelle intact or fragmented. Cell membrane fragments may be derived from the plasma membrane of a cell, or a sub-cellular compartment. The cell membrane or fragments thereof may be produced as a crude extract or they may be purified.

The cell membrane constituents may be in the form of liposomes derived from cell membrane fragments. Cell membrane constituents may also be provided as liposomes derived from purified components, for example, detergent solubilised proteins and synthetic lipids may be additionally provided for incorporation into the bilayer.

In addition to, or alternatively to, the outer cell membranes, cell membranes of sub-cellular structures or organelles, for example mitochondrial membranes and endoplasmic reticulum, may be incorporated into the bilayer.

The cell membrane is, in a beneficial embodiment, eukaryotic. As noted above, it has not previously been the case that a eukaryotic membrane has been easily and directly incorporated into an artificial bilayer. Surprisingly, the present invention provides a method of achieving this.

It is also possible in the present invention that prokaryotic cell membrane may be incorporated into the bilayer. It is possible in the present invention that viral components such as viral envelope proteins may be incorporated into the bilayer.

The cell membrane may be derived from a direct preparation of primary cells. The cell membrane may be derived from tissue harvested from a eukaryote, such as clinically-derived mammalian tissue (e.g. heart tissue, brain tissue, nervous tissue, liver tissue, kidney tissue, blood cells). The tissue may comprise healthy tissue or pathological tissue implicated in disease (e.g. cancerous tissue, damaged heart tissue, blood cells).

Membrane proteins, such as eukaryotic membrane proteins, including oligomeric proteins and single-polypeptide proteins, for example, ion channels; receptors; pores; antigens; enzymes; and transporters may be incorporated into the bilayer via the incorporation of the cell membrane. The membrane proteins may be peripheral to the membrane, i.e. penetrating only a certain extent of the lipid bilayer, or trans-bilayer embedded, where this can mean monotopic or polytopic penetration of the lipid bilayer

The cell membrane comprising cell membrane constituents may comprise proteins, such as membrane proteins. The cell membrane comprising cell membrane constituents may comprise one or more ion channels. A cell membrane derived from a eukaryote, such as a mammalian cell, may comprise endogenously expressed membrane proteins, and/or protein expressed in cells by recombinant means, or a protein expressed from a viral nucleic acid. The protein of interest may be overexpressed in the plasma membrane or sub-cellular membrane or organelle membrane of the cell.

A cell membrane derived from a prokaryote may comprise endogenous membrane protein or exogenous membrane protein expressed by recombinant means, such as a eukaryotic membrane protein.

A cell membrane derived from viral components such as a viral envelope may comprise a viral protein, or a protein derived from a host cell of the virus or other membrane constituents from either host or virus.

The cell membrane may comprise endogenously expressed, or expressed by recombinant means, membrane proteins from any eukaryotic cell line, such as any mammalian cell line, for example, HEK293, SupT1, CHO, Jurkat cells or insect cells such as Sf9, as well as single cell eukaryotes such as yeasts (S. cerevisiae and others).

Expression levels of an endogenous membrane protein may be modulated to up-regulate or down-regulate the amount or concentration of the protein in the cell membrane. Expression of the protein may be modulated by preparing cell cultures under particular growth conditions, or through the addition of agents which modulate gene expression levels. Expression of the protein may be modulated by manipulation of the cell environment, such as the temperature or concentration of environmental gases or chemicals.

Cell membrane constituents may be direct incorporated from intact whole cell membrane.

Cell membrane constituents may be incorporated from cell membrane fragments. Cell membrane fragments may be created by any of the following well-known means:

### 1. Hypo-osmotic Lysis

### 2. Freeze-thawing

### 3. Sonication

### 4. Mild-Detergents

### 5. Enzymatic Digestion of Membranes

### 6. Mechanical Disruption of Membranes

7. Biological generation of membrane fragments

These techniques are described in more detail below.

In the monolayers formed in step (a) of the method of the invention, the amphipathic molecules are aligned on the surface of the hydrophilic body and the hydrated support with their hydrophilic groups (or “heads”) towards the water interface and their hydrophobic groups (or “tails”) away from the water interface. The orientation of the amphipathic molecules in the monolayers means that a bilayer forms when the monolayers are brought into contact.

The amphipathic molecules used in the invention may be lipid molecules; in particular, surfactant molecules may be used. The lipid molecules may be selected from the group comprising fatty acyls, glycerolipids, glycerophospholipids, sphingolipids, sterols, prenol lipids, polyketides, phospholipids and glycolipids. The lipid may be derived from a cell membrane.

The lipid may include any of the group comprising monoolein; 1,2-dioleoyl-sn-glycero-3-phosphocholine (DOPC); 1,2-diphytanoyl-sn-glycero-3-phosphatidylcholine (DPhPC); palmitoyl oleoyl phosphatidylcholine (POPC); 1-palmitoyl-2-oleoyl-phosphatidylethanolamine (POPE); 1-palmitoyl-2-oleoyl-phosphatidylethanolamine; and 1-palmitoyl-2-oleoylphosphatidylglycerol (POPE/POPG) mixtures; or mixtures thereof.

Fluorescent and non-fluorescent probes, may be incorporated into the hydrophobic or hydrophilic media. For example, a lipophilic fluorescent potentiometric dye such as di-8-ANEPPS may be incorporated into the bilayer via the aqueous hydrophilic body and/or hydrated support via vesicles.

The amphipathic molecules in the monolayers may be of the same or different types. For example, each monolayer of the bilayer may comprise a different type of amphipathic molecule such that the bilayer produced is asymmetric.

Alternatively, each monolayer may comprise the same type or mixtures of types of amphipathic molecules.

Hydrophobic compounds may be added to the hydrophobic medium or the hydrated support or hydrophilic body, including, for example, lipophilic agonists and antagonists for membrane protein function modulation.

The hydrated support may comprise a solid or a semi-solid substrate. The terms “solid” and “semi-solid” as used herein are understood to have their ordinary meaning to a person skilled in the art. Essentially the term “solid” refers to a substrate that is rigid and resistant to deformation, and “semi-solid” refers to a substrate that has properties between those of a solid and a liquid. Preferably a semi-solid substrate has some degree of flexibility but is rigid enough to maintain its shape when placed in a container, and will not immediately conform to the shape of the container. An example of a semi-solid substrate is a gel.

The hydrated support may be hydrophilic. The hydrated support may comprise any hydrophilic substrate which is capable of maintaining a layer of water under a solution of lipid-in-oil. Alternatively, the hydrated support may not be hydrophilic and the lipid monolayer may be formed by the attachment of lipid molecules to the surface, for example, the surface of the support may be such that modified lipids will react with the surface and attach to form a monolayer.

The hydrated support may comprise a hydrophilic gel. Preferably a monolayer of amphipathic molecules will self assemble on the surface of the hydrated support if it is placed in the presence of amphipathic molecules in a hydrophobic medium. The hydrated support may be porous or non-porous. Preferably the hydrated support is porous. The hydrated support may be a hydrogel. The hydrogel may be chemically or photo-crosslinked. The hydrated support may be partially or substantially transparent. Alternatively, the hydrated support may be largely opaque.

The hydrated support may be a protein or analyte separation gel, for example, an electrophoresis gel. The separation gel may contain proteins, DNA or other samples separated, for example, on the basis of their size, molecular weight or ionic properties.

The hydrated support may comprise a substrate of any thickness, preferably from about 1 nm to about 10 cm, more preferably from about 1 μm to about 1 cm, most preferably from about 100 μm to about 1 cm.

The hydrated support may comprise any of the group selected from crude agar, chitosan, gelatine, agarose, polyacrylamide, cross-linked polyethylene glycol, nitro-cellulose, polycarbonate, anodise material, polyethersulphone, cellulose acetate, nylon, Naphion materials, mesoporous silica, water, and glass, or combinations thereof.

The hydrophilic body may be a liquid, solid, or semi-solid or a mixture thereof. Preferably the hydrophilic body comprises a droplet of aqueous solution, such as water. Where the hydrophilic body is a droplet of an aqueous solution it preferably has a diameter of from about 5 nm to 10 cm or more, preferably from 1 μm to 1 mm. In one embodiment, droplets are around 100 μm in diameter. The hydrophilic body may comprise a hydrated solid or semi-solid support/substrate. The hydrophilic body may comprise a hydrogel, such as hydrated agarose. The hydrophilic body may comprise an aqueous cell membrane liposome.

The composition of the hydrophilic body and/or the hydrated support may be controlled to contain the correct salts to allow an electrical current to be carried, for example, NaCl, KCl, MgCl, and/or other salts may be included. The hydrophilic body may also comprise common buffering agents to control pH, for example, Bis-tris, Tris, Hepes, sodium phosphate and/or potassium phosphate. Salts may also be included for other reasons, for example, to stabilise proteins, to control electrostatic interactions, to control the osmotic gradient across the bilayer and/or to activate fluorescent probes.

The hydrophilic body and/or hydrated support may comprise varying amounts of other components, such as, agonists or antagonists for membrane protein function, or protein cofactors, sugar e.g. sucrose, or polymers which may be used to stabilise osmotic stresses, amino acids, peptides, nucleic acids, fluorescent or non-fluorescent probes, chemical agents/drugs, dyes, microspheres or beads. The hydrophilic bodies may also comprise denaturants such as urea or guanidine HCl. The other/additional components may be added prior to or following bilayer formation and/or cell membrane incorporation.

The hydrophilic body and/or hydrated support may comprise low-solubility small molecule compounds in trace quantities of organic solvents, mixed with water, for example, mixtures of ethanol, methanol, DMSO, DMF, or chloroform with water.

The hydrophobic medium may be an oil. The oil may be a hydrocarbon, which may be branched or unbranched, and may be substituted or unsubstituted. For example, the hydrocarbon may have from 5 to 20 carbon atoms, more preferably from 10 to 17 carbon atoms. Suitable oils include alkanes or alkenes, such as hexadecane, decane, pentane or squalene, or fluorinated oils, or silicone based oils, or carbon tetrachloride. In one embodiment the oil is an n-alkane.

The hydrophobic medium may be a lipid in oil solution, i.e. amphipathic lipid molecules are provided in the oil. Preferably the lipid in oil solution contains from about 1 mg/ml to about 30 mg/ml of lipid in the oil. Preferably, the lipid in oil solution contains about 5 mg/ml of lipid.

Preferably the lipid/oil solution comprises a phospholipid, such as a phosphocholine lipid, e.g. 1,2-diphytanoyl-sn-glycero-3-phophocholine (DPhPC), in an n-alkane, such as a C10 to C17 n-alkane, e.g. n-hexadecane (C₁₆).

A third phase may be provided by the provision of molecules that are both hydrophobic and oleophobic, such as fluorinated oils, or hydrophobic and oleophobic lipids.

An additional protein may be inserted into the bilayer in addition to any proteins provided by the cell membrane. The protein may be a known membrane protein or alternatively a water-soluble protein. The additional protein may have been purified. The additional protein may be inserted as according to the method disclosed in previous patent publication number WO2009024775.

The bilayer may be used to detect compounds/analytes which interact with amphipathic molecules in the bilayer or with a membrane-associated protein in the bilayer. The interaction with the protein or the amphipathic molecules may be by the specific or non-specific translocation of the analytes/compounds across the bilayer, this may be mediated by the protein or other membrane constituents such as sugars etc. or by the amphipathic molecules. Alternatively compounds/analytes may interact with the protein or with the bilayer to cause physical, optical, electrical, or biochemical changes. Such interaction may be detected in many different ways, including, but not limited to, by visual changes, changes in specific capacitance, or by the activation of fluorescently labelled molecules in or near the bilayer.

The bilayer having incorporated membrane constituents may be used to study processes occurring at, in or across the bilayer. The bilayer may be used to study membrane constituent behaviour. The bilayer may be used to detect proteins or molecules which interact with the membrane constituents reconstituted in the bilayer.

The bilayer may be used to investigate and/or screen cell membrane-proteins; to investigate and/or screen for analytes that interact with the reconstituted membranes; and/or to investigate and/or screen for compounds that interact with bilayers with reconstituted membranes derived from different cell types.

The bilayer may be used to study the ability of a membrane-associated protein in the bilayer to transport molecules across the membrane. For example, the amount of a molecule being transported across a membrane, either through a protein or simply non-mediated transport across the bilayer, may be determined by using voltage studies, mass spectrometry, enzymatic techniques, such as ELISA, or by using a fluorescently or radioactively labelled substrate.

The bilayer may be used to study the effects of mechanical changes of the bilayer on proteins in the bilayer or on the bilayer itself. Mechanical changes which can be studied include, for example, changes in membrane curvature, lateral forces, surface tension etc.

A compound/analyte to be tested, studied and/or used in a screen may be introduced to the bilayer by placing it in the hydrophilic body and/or in the hydrated support and/or in the hydrophobic medium. If included in the hydrophilic body the compound/analyte may be incorporated when the hydrophilic body is formed or it may be added later, for example, by injection into the formed hydrophilic body. Similarly, if the compound/analyte is in the hydrated support it may be incorporated when the hydrated support is formed or added later.

In one embodiment the hydrated support may be a protein separation gel or membrane containing proteins (analytes/compounds) for analysis. For example the sample may be a polyacrylamide gel containing proteins which have been separated on the basis of size. In this embodiment the analytes/compounds are introduced to the bilayer via the hydrated support.

The analyte/compound may be a purified protein or a crude protein extract.

The analytes/compounds to be tested may be in a sample. The sample may be, for example, a sample of blood, urine, serum, saliva, cells or tissue. The sample may be a liquid from a cell growth medium.

The bilayer may be visualised through the hydrated support by optical means for example with an inverted microscope or in some circumstances even by the naked eye. The visualisation of the lipid bilayer may be used to track the formation, position, size, or other property of the bilayer. Visualisation of the bilayer allows labelled analytes/proteins/compounds/cell membranes at or in the bilayer to be seen and studied.

One or more detection means may be used to detect chemical, biochemical, electrical, optical, physical and/or environmental properties of the bilayer of amphipathic molecules or of membrane-associated proteins inserted into the bilayer. In particular the one or more detection means may be used to detect changes in or at the bilayer induced by the compounds/analytes.

The detection means may comprise electrodes which may be used to detect changes in ionic current passing through a protein channel in the bilayer or the electrochemical properties of molecules in the hydrated support, hydrophilic body or bilayer. Currents may be recorded using a standard patch-clamp amplifier or other low noise electrical amplification system.

Chemical or biochemical changes may be detected using enzymatic assays or immunoassays. Alternatively, the use of labelled, for example radio or fluorescently labelled proteins which are activated under certain conditions can be used to monitor changes at the bilayer. Colorimetric methods that respond to changes in light absorption upon reaction may also be used to detect changes in the bilayer, in particular this method may be used to detect a change in the size of the bilayer.

The detection means may be capable of constantly or intermittently detecting properties of, or changes at, the bilayer.

Detection reagents, like membrane-associated proteins, analytes and other compounds may be delivered to the bilayer by incorporation into the hydrophilic body and/or the hydrated support, injection directly into the hydrophilic body and/or the hydrated support, and/or incorporation in, or addition to, the hydrophobic medium. Injection into the hydrophilic body and/or the hydrated support may be achieved using a micropipette.

In an embodiment where changes in membrane capacitance are being studied electrodes may be used as the detection means. The electrodes may be Ag/AgCl, such electrodes may be from approximately 10 microns to 1 mm in diameter. A first electrode may be electrically contacted with the hydrophilic body and a second electrode may be electrically contacted with the hydrated support. Electrical properties of the bilayer, such as the specific capacitance of the bilayer, may be determined using the electrodes.

Fluorescence imaging and intensity measurement may be used to measure the properties of bilayer embedded membrane proteins. For example, the inclusion of potentiometric dyes (such as di-8-ANEPPS) may be used to measure the transbilayer voltage in the presence of ion channels, pores or transporters and or other membrane proteins, and/or function modulators thereof.

Membrane protein function in the bilayer may be investigated by use of any of the group selected from Infra-red spectroscopy, Raman spectroscopy, Surface Plasmon Resonance, Atomic Force Microscopy, SAXS and Neutron Scattering, Differential Scanning Calorimetry, Isothermal Titration Calorimetry, Circular Dichroism Spectroscopy, Electron Microscopy and Mass Spectrometry, or combinations thereof.

Optical and/or electrical characterisation advantageously permits rapid screening of a very large number of membrane proteins or compounds. Optical and/or electrical characterisation also advantageously permits rapid screening of a very large number of bilayers.

The physical, chemical or electrical environment of the bilayer may be controlled by the introduction, removal, or sequestering of reagents, analytes, compounds and/or proteins to or from the bilayer and/or hydrophilic body and/or hydrated support, e.g. the pH of the environment surrounding the bilayer may be controlled by the addition of a buffer to the hydrophilic aqueous body and/or the hydrated support.

Once formed, a bilayer made according to the invention may be translocated or moved across the surface of the hydrated support. Preferably this is achieved by moving the hydrophilic body across the surface of the hydrated support. Preferably incorporated membrane proteins within the bilayer do not disassociate from the bilayer when the bilayer is translocated across the surface of the hydrated support.

The translocation of the bilayer across the surface of the hydrated support may be achieved by moving a member contacted with the hydrophilic body or the hydrated support. The member may be an electrode. The translocation of the bilayer across the surface of the hydrated support may be achieved by microfluidic pumping, in order to effect the translocation of the hydrophilic bodies and therefore the bilayers across the hydrated support.

The translocation of the bilayer may be used to apply forces to proteins in the bilayer, for example to study mechano-sensitive protein channels or to study the effect of such force on the properties of the bilayer itself.

The translocation of the bilayer may be used to scan across the surface of the hydrated support to identify analytes/compounds in the hydrated support which alter properties of the bilayer and/or membrane-associated proteins in the bilayer.

The surprising capability of the bilayer to translocate across the surface of the hydrated support provides the benefit of being able to scan across the hydrated support with the bilayer to detect analytes/compounds, such as proteins and/or reagents or substrates located at different regions of the same hydrated support. This advantageously can be done without having to disassemble the bilayer between each sampling region.

The bilayer may be translocated across a hydrated support comprising an array or library of different compounds. The different compounds may be spotted onto the support in predetermined positions. Alternatively the hydrated support may comprise a separation gel or membrane, containing compounds such as proteins or DNA or small molecules, which have been separated on the basis of their size or ionic properties.

The translocation of one or more bilayers may allow a bilayer comprising one or more particular membrane-associated proteins in a cell membrane to be rapidly screened against a library of compounds in the hydrated support. The screen may allow compounds in a library which interact with a membrane-associated protein and cause a detectable change in properties at the bilayer to be detected. The compounds in the library may be proteins, DNA or other small molecules. The detectable change may be, for example, a change in conductance or a change in fluorescence or other marker pattern.

The bilayer may be formed on a suspended hydrated support in a mobile device, such as a pipette tip, which may then be scanned across a surface of a cell. Alternatively the mobile device may be used to probe different solutions, for example, different biological samples.

Once formed, a bilayer made according to the invention may be increased or decreased in size. The increase or decrease in size of the bilayer may be effected by moving the centre of the hydrophilic body towards or away from the hydrated support.

A plurality of separate bilayers may be formed between a plurality of separate hydrophilic bodies and one or more hydrated supports. The hydrophilic bodies may be arranged in a two, or three, dimensional array. Two or more separate hydrophilic bodies on the same hydrated support may comprise the same or different detection means and/or different reagents, and/or a cell membrane derived from the same cell type or different cell types. An array of aqueous droplets may be deposited over a hydrated support surface to detect the location of analytes/compounds in the hydrated support, for example by the fluorescence of a hydrophilic body or a change in recorded conductance when an analyte/compound is detected.

The bilayer may be formed in a microfluidic channel. The microfluidic channel may comprise an aqueous liquid, which may at least in part provide the hydrophilic body. The microfluidic channel may additionally comprise the hydrophobic medium. At least one wall/floor of the microfluidic channel comprises the hydrated support.

According to another aspect the invention provides a bilayer product comprising a hydrophilic body and a hydrated support immersed in a hydrophobic medium with a bilayer comprising amphipathic molecules between the hydrophilic body and the hydrated support, characterised in that the bilayer incorporates at least part of a cell membrane.

The cell membrane present in the bilayer may be from whole cells and/or from cell membrane fragments.

Preferred embodiments for the hydrophilic body, the hydrated support, the amphipathic molecules and the reconstituted cell membrane are as discussed above.

The bilayer may be formed by the method of the invention.

According to another aspect of the invention, there is provided the use of a bilayer product according to the invention in conjunction with fluorescence microscopy.

The nature of the hydrated support preferably allows the bilayer to be viewed using a microscope. Thus in this aspect of the invention the hydrated support may be a layer no more than about 2 mm thick. The hydrated support may be from about 1 nm to about 2 mm thick, alternatively from about 100 nm to about 1 mm thick, alternatively the hydrated support may be from about 100 nm to about 400 nm thick.

Molecules to be observed may be fluorescently labelled with fluorophores.

Fluorophores in the hydrophilic droplet and/or hydrated support may be observed using total internal reflection fluorescence microscopy or other wide-field fluorescence microscopy techniques. Total internal reflection fluorescence microscopy may be expedited by either objective-type or prism-based geometries. Observations using total internal reflection fluorescence microscopy may be used in combination with electrical measurements. Total internal reflection fluorescence (TIRF) microscopy may be used to observe fluorescence from entities present in the bilayer either as a bulk property of the bilayer, or with suitable detection down to the level of individual molecules. TIRF techniques may be used in combination with other analysis techniques, for example, in combination with acquiring electrical data.

An advantage of using total internal reflection fluorescence to observe the fluorophores is that only fluorophores within about 200 nm of the bilayer are illuminated and thus observed, whilst other fluorophores not close to the cell membrane bilayer are not illuminated and not observed. Using total internal reflection fluorescence measurements in combination with electrical measurements has an advantage that protein-protein interactions can be studied, for example, the electrical response of ion channels to the binding of a fluorescent substrate can be studied.

According to a yet further aspect of the invention there is provided a method of screening for an interaction between a bilayer of amphipathic molecules comprising reconstituted cell membrane constituents and one or more compounds in a library comprising:
i) providing a bilayer product according to the invention;
ii) translocating the hydrophilic body and thus the bilayer across the surface of the hydrated support; and
iii) detecting any interaction between the bilayer and a compound in the hydrated support and/or hydrophilic body.

The cell membrane constituents may be as described herein, for example intact or fragments of cell membranes reconstituted in the artificial bilayer.

The hydrated support and/or the hydrophilic body may comprise the library of compounds to be tested.

Translocation of the bilayer may be achieved by the direct or indirect contact of the hydrophilic body with a micromanipulator arranged to move the hydrophilic body. The hydrophilic body may be in contact with an electrode which may be moved to translocate the bilayer across the hydrated support.

The method of screening may be automated to allow high throughput screening to be undertaken.

According to another aspect of the invention, there is provided the use of a bilayer product according to the invention to identify substances capable of interaction with a cell membrane derived membrane-associated protein and/or other membrane constituents located in the bilayer.

The bilayer may be used in pharmaceutical drug development or in medical diagnostics for primary-cell screening. For example, a target compound or substance may be incorporated either in the hydrophilic body or hydrated support, and the functional effects of such a compound or substance on a protein in the bilayer may be probed.

The interaction or effects may be translocation of the substance or the target compound, modulation, such as inhibition or activation, of protein function by the substance or the target compound. The interaction or effects may be a conformational change in the protein(s).

It will be appreciated that all the optional and/or preferred features of the invention discussed in relation to only some aspects of the invention may be applied to all aspects of the invention.

Embodiments/aspects of the invention will now be described by way of example only with reference to the accompanying figures, in which:

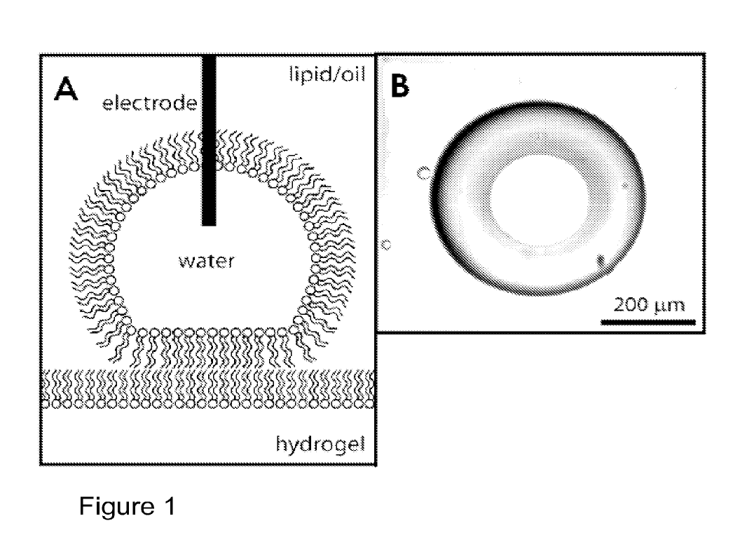

FIG. 1—illustrates an aqueous droplet (hydrophilic body) on a hydrated support bilayer, referred to herein in as a droplet-on-hydrated-support bilayer (DHB); FIG. 1A is a diagram of a droplet-on-hydrated-support bilayer. A lipid monolayer spontaneously forms on the aqueous surface of the aqueous (water) droplet and the hydrated support (hydrogel) when each is immersed in a solution of lipid in hydrophobic oil. When the monolayers of the two components are brought into contact they form a lipid bilayer; FIG. 1B shows a droplet bilayer (DHB) visualised from below with an inverted microscope—the image shows a droplet, without an electrode, supported on a hydrogel surface. The single continuous bilayer area in the centre of the droplet is easily seen due to the large change in refractive index at the interface;

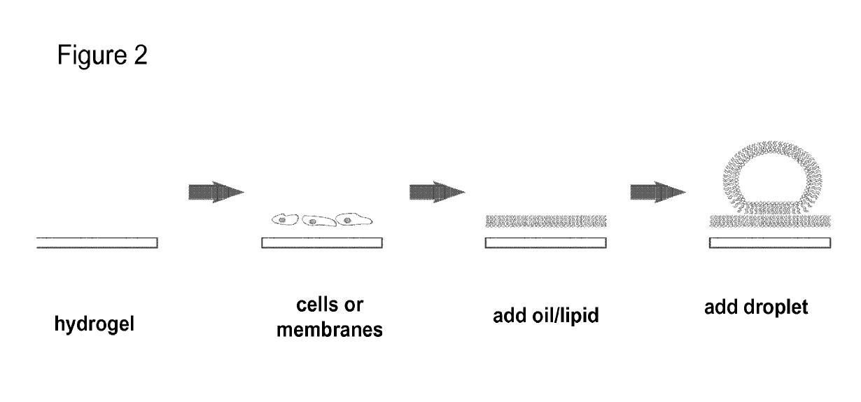

FIG. 2—shows a schematic diagram of incorporation of a cell membrane into a bilayer according to the invention;

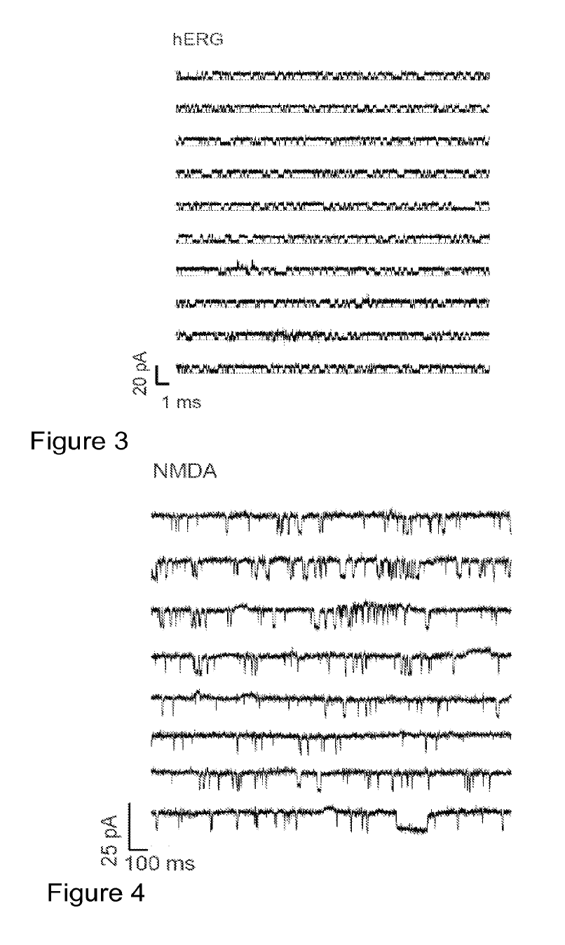

FIG. 3—shows the result of a single channel recording of hERG channels in HEK293 cell membrane incorporated into a bilayer;

FIG. 4—shows the result of a single channel recording of NMDA channels in 3T3 cell membrane incorporated into a bilayer;

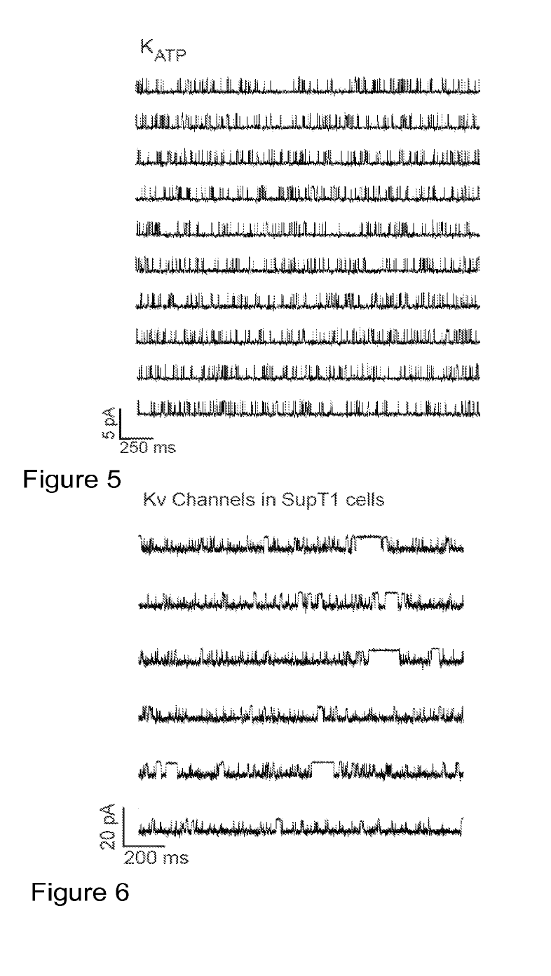

FIG. 5—shows the result of a single channel recording of K_{ATP} channels in Min6 cell membrane incorporated into a bilayer;

FIG. 6—shows the result of a single channel recording of a Kv channel in SupT1 cell membrane incorporated into a bilayer;

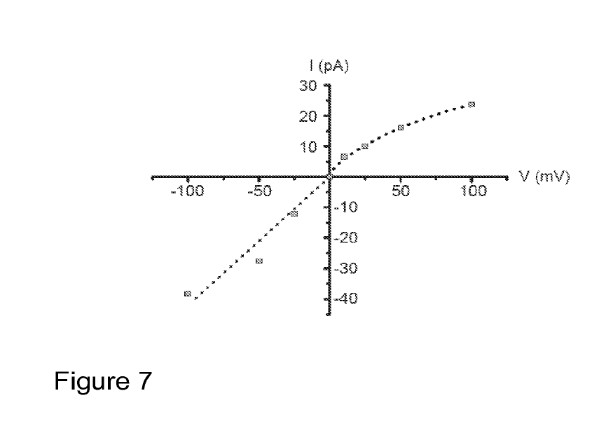

FIG. 7—shows a Kv channel I-V curve from SupT1 cells discussed in FIG. 6;

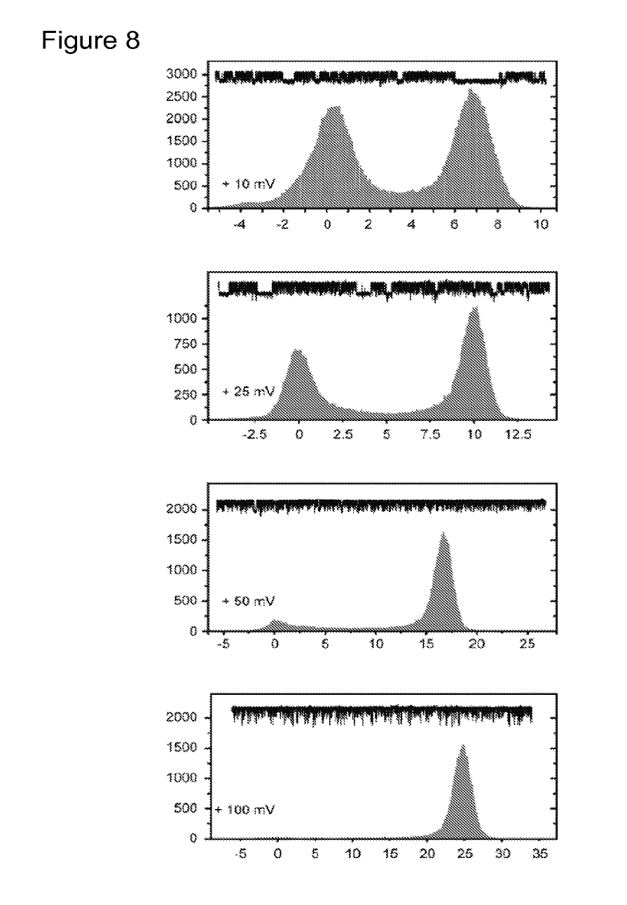

FIG. 8—shows voltage dependence of the Kv channels discussed in FIGS. 6 and 7 on open probability;

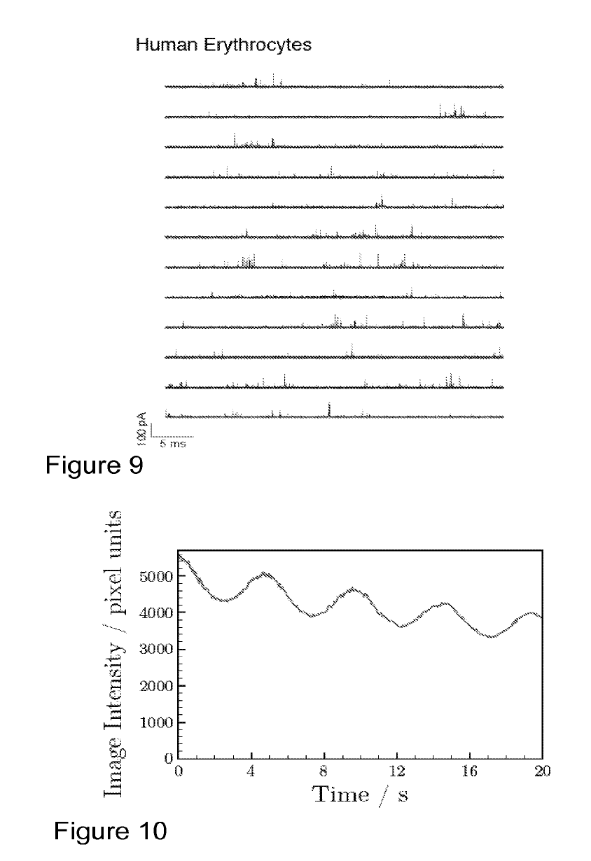

FIG. 9—shows the result of a single channel recording of human erythrocyte derived cell membrane bilayer;

FIG. 10—illustrates use of fluorescent membrane potentiometric probes in a bilayer, where an alternating externally applied potential difference drives a concomitant fluorescence intensity change.

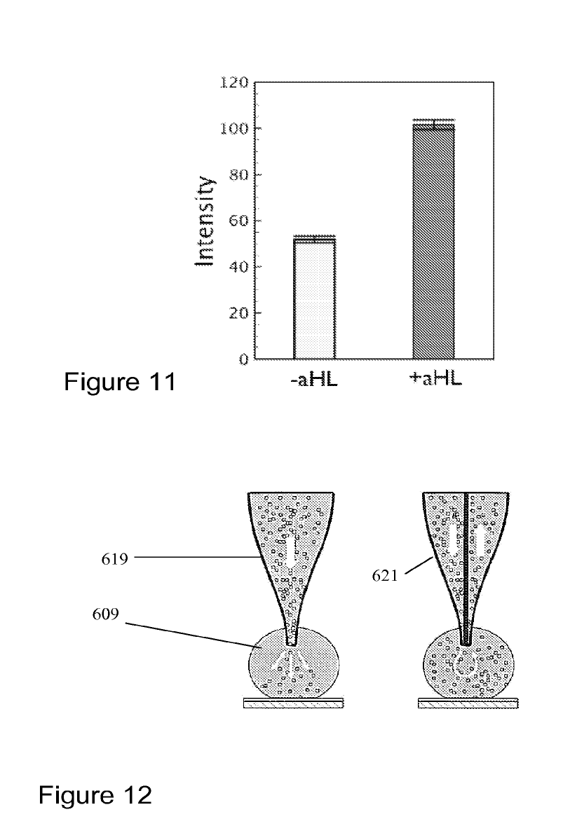

FIG. 11—illustrates another use of fluorescent membrane potentiometric probes in a bilayer.

FIG. 12—shows a diagram of how reagents can be introduced into an aqueous droplet by injection from (A) a micro-pipette, or (B) a micro-pipette with a multi bore capillary;

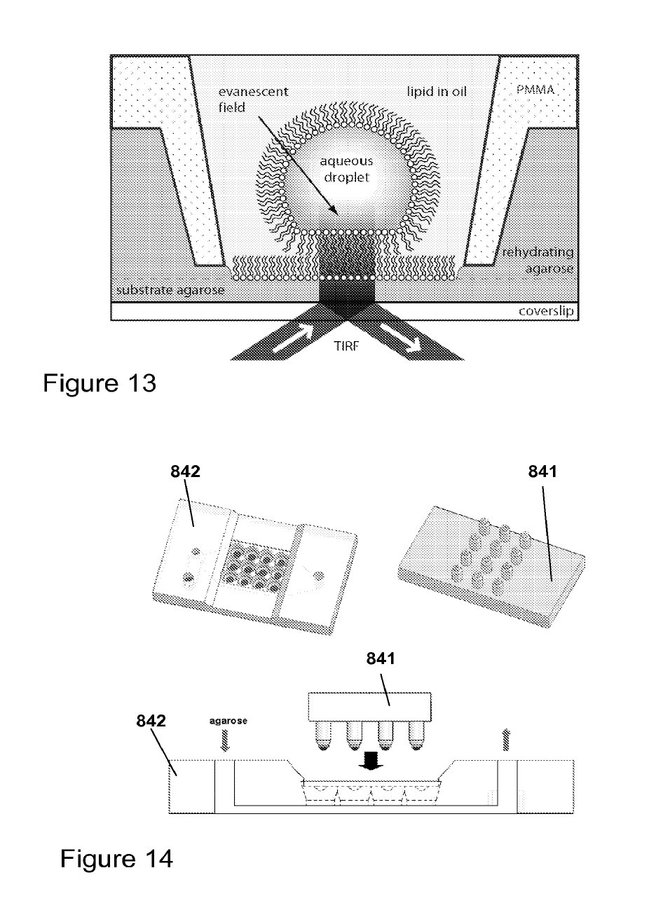

FIG. 13—illustrates total internal reflection fluorescence microscopy on a droplet-on-hydrated-support bilayer; and

FIG. 14—shows a device for producing an array of bilayers.

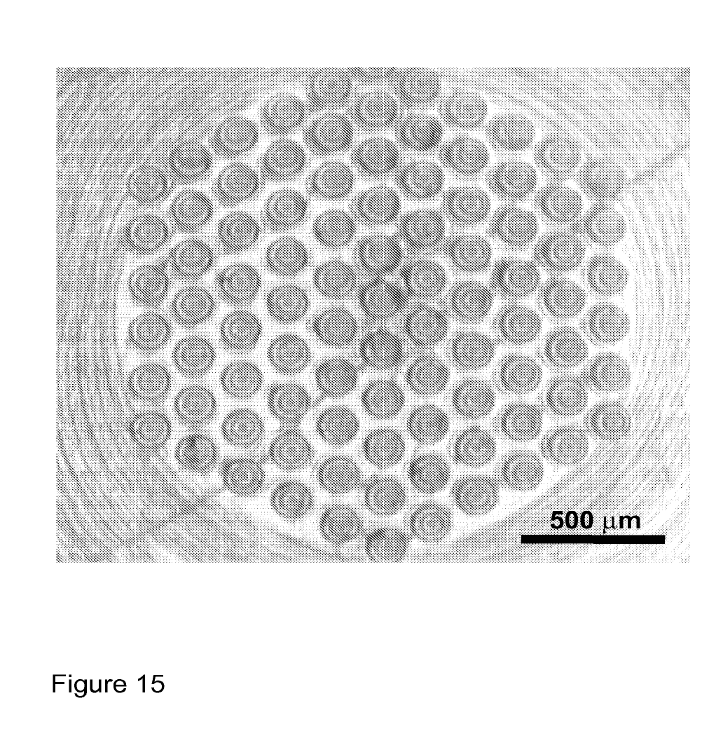

FIG. 15—shows an array of bilayers formed with a hexagonal PDMS stamp, used for experimental screens. 150 mm diameter wells were fabricated resulting in a bilayer density of 360 bilayers/cm².

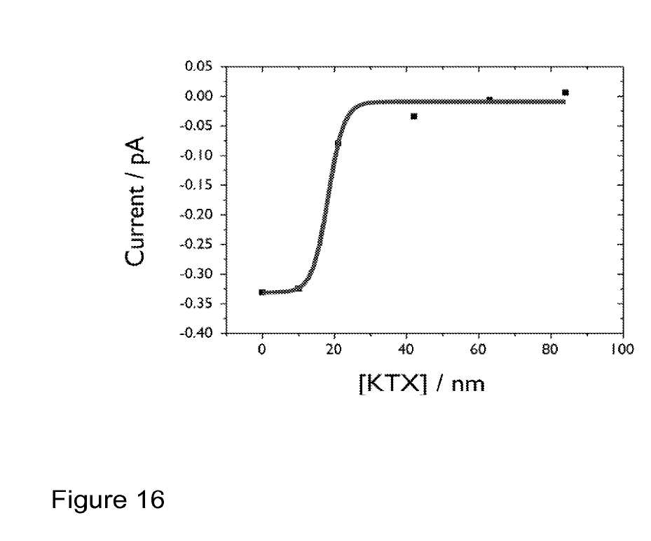

FIG. 16—shows the result of a current block upon titration of kaliotoxin (KTX) with KcsA-ChiIV;

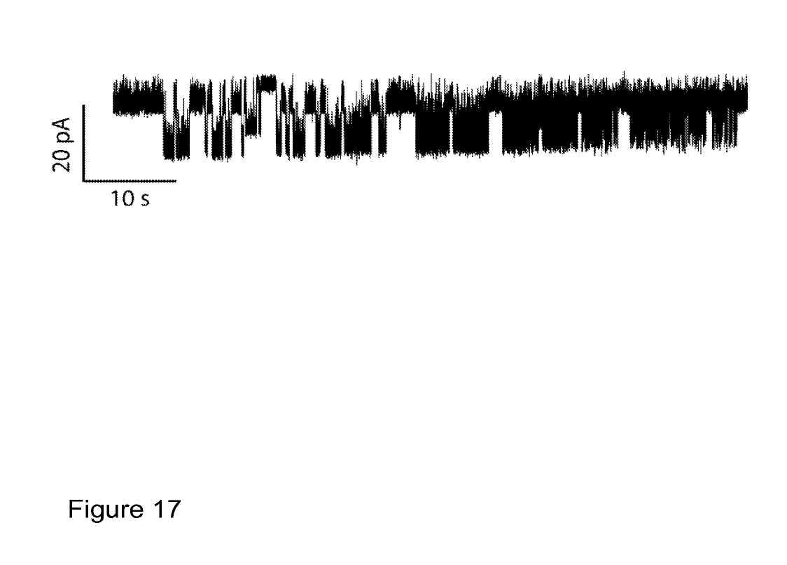

FIG. 17—shows the result of a single channel recording of a KcsA-ChiIV from bacteria reconstituted into the bilayer directly from the E. coli host membrane;

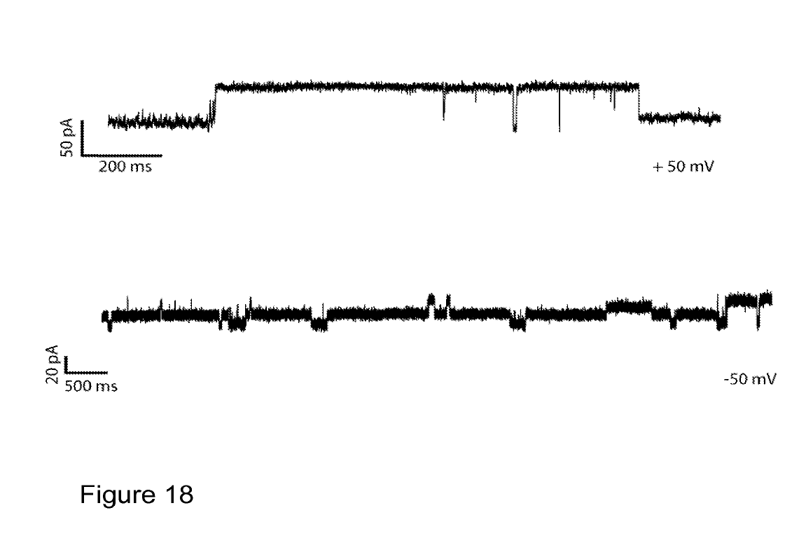

FIG. 18—shows the result of single channel recordings at positive and negative potentials for mitochondrial channels;

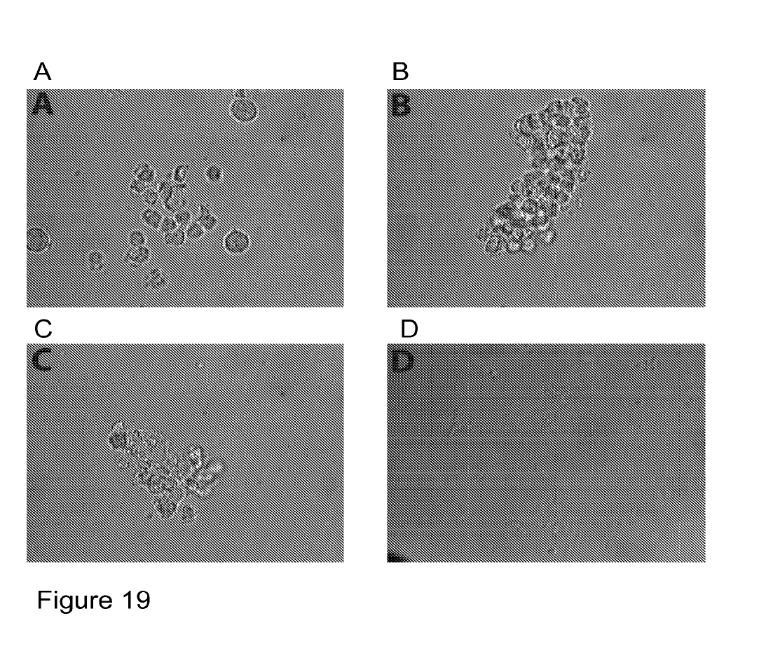

FIG. 19—shows the result of an enzymatic digestion of the membrane of SupT1 cell with phospholipase C. A: SupT1 cells in DMEM B: SupT1 in DMEM with Phospholipase C C: SupT1 in DMEM with Phospholipase C, sonicated (2 mins) D: SupT1 in DMEM with Phospholipase C, sonicated (2 mins), Freeze-thaw (3×);

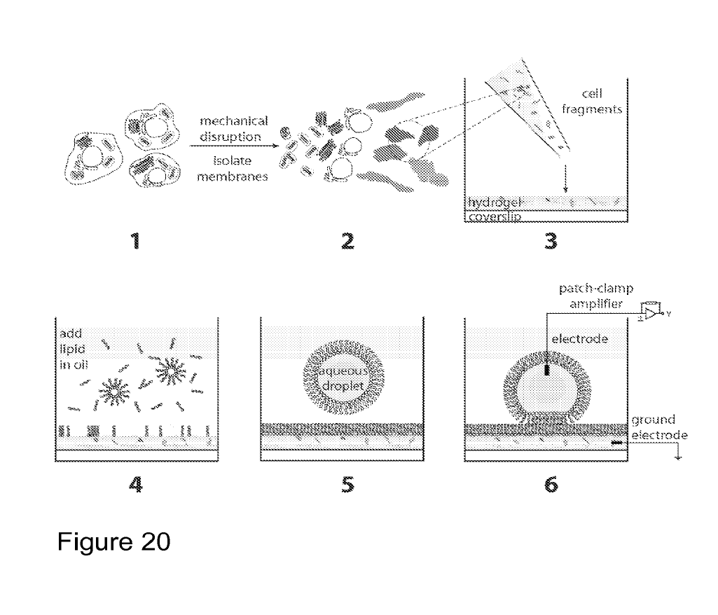

FIG. 20—illustrates schematically the reconstitution system used to incorporate eukaryotic ion channels into a lipid bilayer according to the invention. (1) Cells are mechanically disrupted and membranes isolated (2). Cell membrane fragments are deposited onto a hydrogel coated coverslip or incorporated into a hydrogel (3). A lipid in oil solution is added and a lipid monolayer forms at the interface (4). An aqueous droplet with a lipid monolayer is added into the oil (5). Both lipid monolayers come together to form a lipid bilayer; reconstitution of ion channels in the cell membrane fragments in the lipid bilayer occurs during this process. An electrode is introduced into the aqueous droplet to measure ion flux across the bilayer (6);

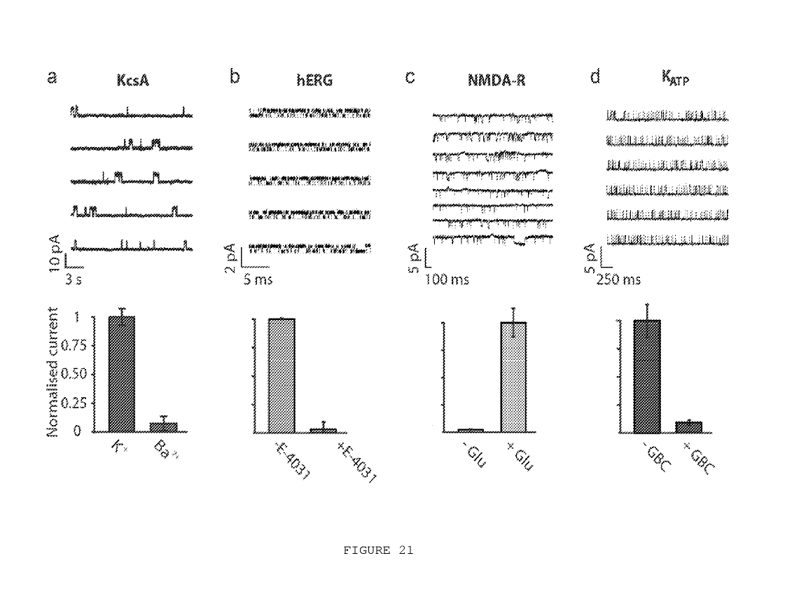

FIG. 21—shows Single Channel Recording (SCR—upper panels) and bulk ion current of ion channels over-expressed in cell lines (lower panels). (a) SCR of KcsA expressed in the porin-free E. coli strain PC2889 (+50 mV in 150 mM KCl, 10 mM Succinic acid, pH 4). The column graph represents bulk current recordings in absence or presence of 150 mM Ba²⁺ (errors bars are standard deviation from 5 experiments) (b) SCR of a hERG channel expressed in HEK293 cells (+100 mV in 350 mM KCl, 10 mM HEPES, pH 7.5). The column graph represents bulk current recordings in absence or presence of the blocker E-4031 (c) SCR of the NMDA receptor from over-expressed in 3T3 cells (140 mM NaCl, 10 mM HEPES, pH 7.5 in droplet and 140 mM NaCl, 10 mM HEPES, 10 μM glycine, 10 μM glutamate, pH 7.5 in agarose, +50 mV). The column graph represents bulk current in the absence and presence of glutamate. (d) SCR of a KATP channel expressed in Min6 cells (140 mM KCl, 10 mM HEPES, pH 7.5, +50 mV). The column graph represents bulk current of KATP channels in the presence and absence of glibenclamide (GBC).

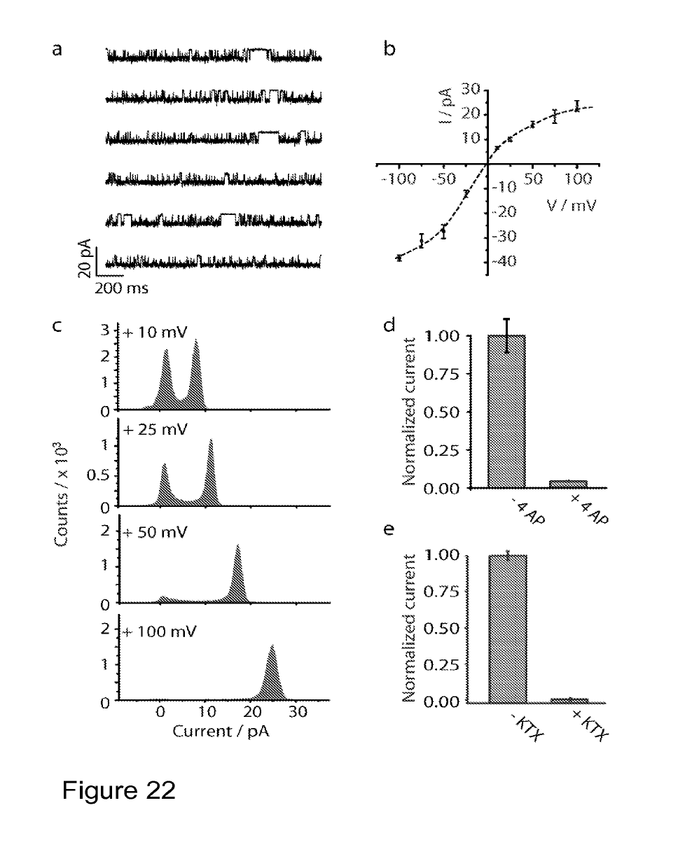

FIG. 22—shows voltage-gated potassium channels in lymphocyte membranes (a) Single channel recording of a K+ channel from lymphocyte membranes reconstituted into a droplet lipid bilayer (150 mM KCl, 10 mM Hepes, pH 7.5, +25 mV). (b) Current-voltage curve of K+ channels obtained from lymphocyte membranes. (c) All-point current histogram showing the voltage dependence of channel open probability. (d) Bulk current block of potassium channels from lymphocyte membranes using 4-AP. In presence of 4-AP ion flux is reduced by 96% compared to a measurement in absence of the blocker. (e) Bulk current block of K⁺ channels from lymphocyte membranes by Kaliotoxin (KTX). In the presence of KTX ion flux is reduced to 99.4% (right) compared to ion flux in absence of KTX (left). Error bars are determined as the standard deviation from 5 experiments;

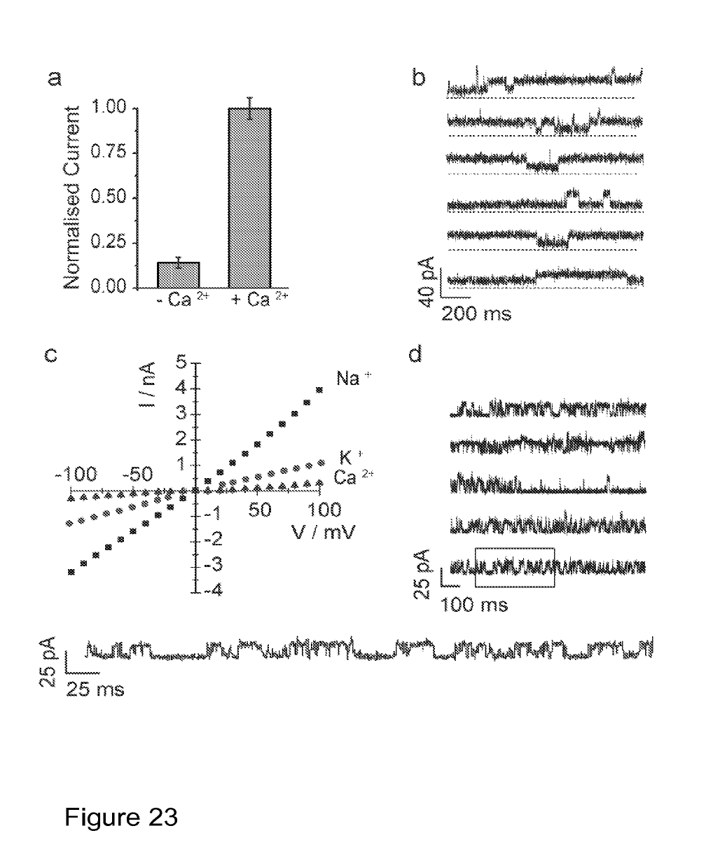

FIG. 23—shows electrical recordings in primary cells and organelles (a) Normalised bulk currents from red blood cell membranes recorded in absence or presence of 5 mM Ca²⁺ (150 mM KCl, Hopes, pH 7, +50 mV). Error bars are determined as the standard deviation from 5 experiments. (b) Single channel recordings of a potassium conductive channel in mitochondrial membranes (150 mM KCl, 10 mM Hepes, pH 7.5, +50 mV) (c) Bulk current recording of reconstituted sickle cell membranes testing the conductance of different cations (Na⁺, K⁺, Ca²⁺). Sickle cell membranes are highly conductive to all cations whereas control membranes from healthy individuals show no conductance. (d) Single channel recordings of a sample of red blood cells with the HbSS genotype, conducting sodium (140 mM NaCl, 10 mM Hepes, pH 7.4, +125 mV). Bottom: enlarged section of (d).

“A droplet-on-hydrated-support bilayer” (or DHB) or “droplet lipid bilayer” as referred to in the specific examples is the same as “a bilayer of amphipathic molecules” as previously discussed. The aqueous body as referred to in the specific examples is equivalent to the hydrophilic body as previously discussed.

### Methods

### Materials

1,2-Diphytanoyl-sn-glycero-3-phosphocholine (Avanti Polar Lipids), hexadecane (Sigma-Aldrich), were used without further purification.

### Creating Droplet-On-Hydrated-Support Bilayers

10 mM 1,2-diphytanoyl-sn-glycero-3-phosphocholine (DPhPC) in hexadecane (C₁₆) was used as the lipid/oil solution in all experiments. Aqueous volumes immersed in this solution spontaneously self-assemble a DPhPC monolayer, and when the monolayers of two components are brought into contact they spontaneously form a lipid bilayer (Tsofina, L. M. et al., 1966. Nature 212, 681-683; Malmstadt, N. et al., 2006. Nano Letters 6, 1961-1965; Funakoshi, K. et al., 2006. Analytical Chemistry 78, 8169-8174; Holden, M. A. et al., 2007. Journal of the American Chemical Society—in press). A droplet-on-hydrated-support bilayer is formed by contacting aqueous droplets with porous hydrated supports such as hydrogels (FIG. 1A). A stabilisation period of at least 5 minutes was required before contacting monolayers to prevent fusion. After this period, bilayer formation was observed with almost 100% efficiency within a few seconds to a minute of contact of an aqueous droplet with the hydrated support. The droplet-on-hydrated-support bilayers were visualised on an inverted microscope (FIG. 1B), this technique was used to track the position of a lipid bilayer during experiments.

The lipid bilayers were electrically accessed by inserting a 100 μm diameter Ag/AgCl wire electrode into the droplets (FIG. 1A) using a micromanipulator. With a corresponding Ag/AgCl ground electrode in the hydrated support, electrical measurements across the lipid bilayer were carried out. Currents were recorded with a patch clamp amplifier (Axopatch 200B; Axon Instruments), and digitized at different frequencies National Instruments Ni-DAQ). Electrical traces were filtered post-acquisition (100 Hz lowpass Gaussian filter) and analyzed using WinEDR. The lipid bilayers were typically able to withstand voltages up to approximately 300 mV while retaining seals in excess of 100 GΩ. Electrical noise levels were typically of the order of ±01.0 pA r.m.s with a 100 kHz recording bandwidth. This reflects the limitations of this apparatus and not the inherent noise in droplet-on-hydrated-support bilayers.

### Droplet-On-Hydrated-Support Bilayers for Fluorescence

Droplet-on-hydrated-support bilayers laid down on thin hydrated supports can be fluorescently examined using total internal reflection fluorescence (TIRF) microscopy.

FIG. 13 illustrates total internal reflection fluorescence microscopy on a droplet-on-hydrated-support bilayer, a supporting substrate comprised of a thin layer of agarose is formed on a glass coverslip. This thin substrate is rehydrated by filling a polymethyl methacrylate (PMMA) micro-channel device with aqueous agarose. The device wells are filled with a solution of lipid in oil. An aqueous droplet is placed on top of the hydrogel underneath the oil. A lipid bilayer forms at the interface between the two aqueous phases. The evanescent field propagates into the droplet-on-hydrated-support bilayer illuminating fluorophores in the lipid bilayer.

Method to Incorporate Membrane Proteins into the Lipid Bilayer
1. A hydrogel such as agarose is deposited on a planar solid support, e.g. a glass slide.
2. A solution containing cell membrane fragments or intact cells is pipetted onto the hydrogel.
3. The hydrogel is immersed in a lipid in oil solution.
4. An aqueous droplet is pipetted into the lipid in oil solution, and upon contact with the hydrogel, a bilayer containing the membrane protein is formed.

In an alternative embodiment, cell membrane may also be incorporated from fragments or cells present in the aqueous droplet rather than the underlying hydrogel.

Due to the giga-ohm electrical seal of a droplet lipid bilayer, single-channel resolution electrical measurements are possible, or alternatively bulk electrical current measurements, similar to whole-cell electrical recording can be made. The planar hydrogel substrate also permits high-sensitivity optical imaging of the bilayer as a means to probe the behaviour of proteins within the bilayer.

Direct Incorporation of Membrane Proteins from Intact Whole Cells

Cell membrane components are incorporated into a bilayer by straightforward incorporation of intact whole cells without any disruption of the membrane. Intact/whole cells are placed in an isotonic medium and incorporated into the aqueous phase of either the hydrogel (hydrated support) or the aqueous droplet (hydrophilic body).

### Preparation of Cell Membrane Fragments

Protein-containing membrane fragments are created by any of the following means:

### 1. Hypo-Osmotic Lysis

Cells are placed in a hypotonic aqueous medium, for example distilled water or hypotonic buffer (less than ˜140 mM total osmolyte), where osmotic pressure exerted from inside the cell will swell the cells and lead to spontaneous membrane disintegration to form cell membrane fragments. Cellular components including all the cytosolic components are then free to diffuse into the suspending medium, in addition to large membrane fragments.

### 2. Freeze-Thawing

Cells are placed in an aqueous/buffering medium, isotonic with the cells, where they are then frozen slowly to at least 0° C. to create water ice and subsequently thawed, repeatedly. The action of the thawing ice crystals is to shear the cell membranes leading to disintegration of the cells to form cell membrane fragments and gradual shrinking of membrane fragments with further repetitions. This process can be tailored (i.e. number of repetitions) to control the size of fragments, and hence the amount of cell membrane protein for reconstitution into the bilayer by dilution.

### 3. Sonication

Similarly to freeze-thawing, cells in an aqueous/isotonic buffered medium are sheared by use of an ultra-sonic bath or ultra-sonic probe. Increased power applied (higher probe frequency, or vibration distance) in the bath decreases the size of the cell membrane fragments as does increased incubation time. This process can be tailored to control the size of cell membrane fragments, and hence the amount of cell membrane protein for reconstitution into the bilayer by dilution.

### 4. Mild-Detergents

Mild lipophilic detergents (e.g. CHAPS, DM, OG) are used to disperse the cell membrane. Once dispersed, remaining protein-containing cell membrane fragments can be diluted to control the amount of protein reconstituted into the DHBs.

### 5. Enzymatic Digestion of Membranes

Phospholipid-digesting enzymes, for example phospholipases, are capable of digesting intact cell membranes to produce smaller protein-containing cell membrane fragments (proteoliposomes). The size of the fragments generated is controlled through careful control of enzyme concentration and duration of incubation, hence affecting the amount of protein reconstituted into the DHBs. For example, FIG. 19 shows the result of an enzymatic digestion of the membrane of SupT1 cell with phospholipase C. A: SupT1 cells in DMEM B: SupT1 in DMEM with Phospholipase C C: SupT1 in DMEM with Phospholipase C, sonicated (2 mins) D: SupT1 in DMEM with Phospholipase C, sonicated (2 mins), Freeze-thaw (3×)

### 6. Mechanical Disruption of Membranes

Pressure-driven devices such as homogenizers and ‘French-Presses’ may be employed to rupture and shrink membranes and fragments generated therefrom. Other such devices which make use of shear forces to disrupt cells, such as for example nozzles, may be used to disrupt cell membranes. Extrusion through nozzles or filters (with known dimensions) can be used to prepare precisely sized membrane fragments.

### 7. Biological Generation of Membrane Fragments

Pore-forming agents (peptides, proteins, polymers) are used in order to perturb the cellular osmotic balance, leading to spontaneous cell disintegration, whereupon membrane fragments containing the membrane protein of interest are reconstituted into a DHB following the removal of the pore-forming agents. Furthermore, a protein can be co-expressed which induces membrane vesicle formation, or cell membrane disintegration, leading to either budding of defined proteolipid vesicles from the plasma membrane or membrane disintegration.

All the methods described above for the manipulation of membrane fragment size are capable of being performed in any combination or individually, depending upon the requirements of the fragments, i.e. higher density protein expression in a target membrane may require greater membrane shearing than for low-levels of target protein expression for accurate dilution of membrane fragments. This facilitates therefore accurate control of the levels of protein reconstituted into DHBs. The preparation methods may be performed in any combination and in any order.

Membrane fragments may be mixed with any aqueous medium containing dissolved lipids, either as micelles or vesicles. This procedure can be performed prior to the methods described above to manipulate the size of membrane fragments.

The resulting fragments of cell membrane, including cellular cytosolic constituents may be separated and harvested by standard biochemical techniques such as centrifugal separation or dialysis (to reduce volume, and/or remove cytosolic components). Resulting preparations of cell membrane fragments can be buffered and incorporated into the DHB by incorporation in either aqueous phase (hydrated support or hydrophilic body) on each side of the DHB. Membrane fragments prepared by these techniques spontaneously incorporate into the lipid bilayer, as determined by monitoring the electrical activity of embedded ion channels carried into the DHB from the fragment preparation.

### Preparation of Cellular Membrane Fractions Containing Target Membrane Protein of Interest

As an example, a cell-line from mammalian cell-culture, for example HEK293 cells, is harvested from culture medium by scraping or trypsin (protease) treatment to detach them from the growth vessel. The cells are washed in an isotonic buffer to remove culture medium by suspending the cells in an excess of buffer and centrifuging the cells at low speed (100×g for 10 minutes). This process is repeated to dilute the remaining cell culture medium in the sample.

In order to prepare a membrane fraction from the intact cells, they are first pelleted as described above. Cells swell and rupture due to osmotic pressure resulting in the generation of large plasma-membrane fragments. Cytosolic constituents of the cells are released into the medium. Then the post-centrifugation medium is replaced by a hypotonic medium and recentrifuged to retain fragments at higher g-forces e.g. 25,000×g for 30 minutes.

The dense membrane fractions and sub-cellular organelles are separated from the medium by repeated exchange of the supernatant during high-speed centrifugation (e.g. 25,000×g). Sucrose gradients may be prepared to separate sub-cellular components by exploiting their different sedimentation properties during high-speed centrifugation. Less dense species (i.e. mitochondria) are separated using this approach from the larger endoplasmic reticulum and plasma membrane.

During the procedure of centrifugal preparation of membrane fragments, the buffering conditions may be altered and the media can be replaced with any desired salt concentration, pH, osmolyte contents, ligands and protein-cofactors, agonists and antagonists of interest.

The sample is then ready for incorporation into the droplet-on-hydrogel lipid bilayer (DHB) system.

### Preparation of Droplet-On-Hydrogel Lipid Bilayer (DHB) Aqueous and Hydrophobic Media

Where it is required that the target protein is inserted from the hydrophilic substrate side of the DHB, the membrane-fragment preparation described above is incorporated by direct addition to warm (˜50° C. cooling down immediately) molten agarose prior to DHB substrate preparation. Such agarose is prepared by melting agarose powder in a desired buffer (most commonly containing an electrolyte e.g. KCl or NaCl and a buffering compound whose pH is titrated with acid or base to the desired value, most often a neutral pH). A small volume of warm agarose (˜50° C.) is added to an equal volume of a desired dilution of membrane-fragment preparation, whereupon rapid cooling ensues. The aqueous agarose membrane fragment mixture is spread across a surface (preferably an optically transparent surface for imaging) for DHB substrate preparation.

### Preparation of DHB Device

The substrate aqueous agarose layer is now ready for submersion under a lipid-in-oil solution of n-decane (e.g. hexadecane C16) with 5-10 mM phosphocholine lipid dissolved in it (e.g. 1,2-diphytanoyl-sn-glycero-3-phosphocholine). A plastic holding device is placed on top of the DHB substrate containing a reservoir of electrolyte-containing aqueous agarose and lipid-in-oil solution. The device is then used to contact aqueous droplets with the substrate hydrophilic aqueous agarose.

### Preparation of Droplets

Aqueous droplets are pipetted (or can be generated by a microfluidic device) into either an identical lipid-in-oil solution as used for the preparation of the DHB device, or alternatively a different lipid-in-oil solution for an asymmetric bilayer. Most commonly a symmetrical bilayer is required, where both bilayer leaflets contain identical lipids. After a short period of equilibration in these lipid-in-oil solutions a monolayer of lipid forms at the interface between the aqueous droplets and the hydrophobic lipid-in-oil solution. Typically this takes less than 5 minutes, but the length of time for equilibration can be affected by lipid concentration in the bathing solution.

### Contact of Lipid-Equilibrated Aqueous Droplets and Lipid-Equilibrated Hydrophilic Agarose Substrate

Droplets are introduced onto prepared lipid-equilibrated DHB substrate agarose by pipetting (alternatively by microfluidic pumping, gravitational sedimentation, or direct injection) into the vicinity of the substrate. A short period of time may be required for a lipid bilayer to form at the interface between the aqueous media (droplet and substrate), for example, approximately one minute.

### Protein Reconstitution Verification

Protein reconstitution into the droplet-on-hydrogel lipid bilayer ensues autonomously as fragments of membrane containing the target protein of interest are spontaneously incorporated into the lipid bilayer at any stage during or after lipid bilayer formation. This process is verified by either electrophysiological recording of ion channel currents present in the DHB or through direct imaging of fluorescent properties associated with the proteins.

### Experimentation

Upon lipid bilayer formation and reconstitution of cell membrane fragments containing the target membrane protein of interest, any electrophysiological protocol can be carried out in order to probe the function of the reconstituted ion channels. Conditions may be prescribed by the chemical constituents of the system. Externally applied voltages across the lipid bilayer may be provided by incorporating electrodes into electrolyte-containing aqueous media either side of the bilayer. The properties of the incorporated proteins may be imaged fluorescently either using potentiometric dyes or other fluorophores. It is obvious to a skilled person familiar with the field that it is possible to perform other biophysical experiments to probe reconstituted membrane protein function, for example by optical techniques such as Surface Plasmon Resonance, Circular Dichroism Spectroscopy, Raman Spectroscopy etc.

Upon establishment of a stable DHB separate chemical components may be incorporated into the system, such as for example a ligand for a membrane protein, i.e. agonists, antagonists, or other chemical species into the aqueous compartments either side of the lipid bilayer by injection directly into droplets or by droplet fusion. For example, this may be used to monitor the behaviour of ion channels prior to the addition of a blocker, where the protein function can be monitored pre and post-addition of an agonist or antagonist.

The following examples demonstrate the incorporation of a wide variety of cell membrane bilayers comprising ion channels as determined by single-channel.

hERG Channels in HEK293 Cells

FIG. 3 shows the result of a single channel recording of a hERG channel overexpressed in HEK293 cells and incorporated into a bilayer. HEK293 cell membranes were washed in 350 mM KCl, 10 mM HEPES, pH 7.5 (Pelleted at 4° C.). A 1:40000 dilution was mixed with substrate agarose (5 uL) before device assembly and submersion in lipid in oil solution (DPhPC in hexadecane). Applied voltage (displayed trace): +100 mV. Buffer: 350 mM KCl, 10 mM HEPES, pH 7.5

### NMDA Channels in 3T3 Cells

FIG. 4 shows the result of a single channel recording of an NMDA channel overexpressed in 3T3 cells and incorporated into a bilayer. NMDA expressing 3T3 cells with approx. 10,000 receptors/cell, were washed by pelleting in 140 mM NaCl, 10 mM HEPES, pH 7.25 (3×). Cells were then subjected a freeze-thaw cycle 3× times (RTP to −80° C.) prior to sonication in a water bath at 4° C. for 3 mins. Sample was mixed to 1:25000 dilution with agarose for substrate agarose layer (5 uL) before device assembly and submersion in lipid in oil solution (DPhPC in hexadecane).

Applied voltage (displayed trace): +50 mV. Buffer: 140 mM NaCl, 10 mM HEPES, pH 7.5 in Droplet. Buffer: 140 mM NaCl, 10 mM HEPES, 10 uM Glycine, 10 uM Glutamate, pH 7.5 in agarose.

### K_{ATP} Channels in Min6 Cells

FIG. 5 shows the result of a single channel recording of a K_{ATP} channel overexpressed in Min6 cells and incorporated into a bilayer. Min6 cells were washed in 140 mM KCl, 10 mM HEPES, pH 7.5 and pelleted and sonicated for 5 mins. A 1:10000 dilution was mixed with substrate agarose (5 uL) before device assembly and submersion in lipid in oil solution (DPhPC in hexadecane). Buffer: 140 mM KCl, 10 mM HEPES, pH 7.5. Applied voltage (displayed trace): +50 mV.

### Kv Channels in SupT1 Cells

FIG. 6 shows the result of a single channel recording of a Kv channel overexpressed in SupT1 cells and incorporated into a bilayer. SupT1 Suspension cells were grown in DMEM, then a solution with 100,000 cells in 20 uL was frozen at −80 C. The cells were subjected to four freeze-thaw cycles to room temperature, then diluted 1:80,000 and pipetted onto a 5 uL substrate agarose layer spread upon a glass coverslip. Applied voltage (displayed trace): +10 mV. Buffer: 150 mM KCl, 10 mM HEPES, pH 7.5

FIG. 7 shows a Kv channel I-V curve from SupT1 cells according to the above experiment. FIG. 8 shows voltage dependence of the Kv channels as per above on open probability. Channels show characteristic increased open probability with increasing voltage.

### Human Erythrocytes

FIG. 9 shows the result of an electrical recording of human erythrocyte derived cell membrane bilayer. Human erythrocytes were washed in 140 mM KCl, 10 mM HEPES, pH 7.5 repeatedly and the serum removed including white cells. Followed by sonication in hypotonic buffer for 5 mins. A 1:100000 dilution was mixed with substrate agarose (5 uL) before device assembly and submersion in lipid in oil solution (DPhPC in hexadecane). Buffer: 2 M KCl, 10 mM HEPES, pH 7.5. Applied voltage (displayed trace): +100 mV.

### KcsA-ChiIV Channel Recording

FIG. 16 shows the result of a current block upon titration of kaliotoxin (KTX) with KcsA-ChiIV and FIG. 17 shows the result of a single channel recording of a KcsA-ChiIV from bacteria reconstituted into the bilayer directly from the E. coli host membrane.

### Mitochondrial Channel Recording

FIG. 18 shows the result of single channel recordings at positive and negative potentials for mitochondrial channels;

### Use of Fluorescent Membrane Potentiometric Probes in a Bilayer

With reference to FIG. 10, a potentiometric dye (di-8-ANEPPS) is incorporated in the lipid in oil solution. It resides perpendicular to the plane of the lipid bilayer in both leaflets parallel with the lipids. Fluorescence from the dye alters according to the potential difference across the lipid bilayer. Here, an externally applied alternating sine wave potential difference is applied across the DHB from between +/−150 mV. The dye can be clearly seen reporting the potential difference across the DHB as the sine wave alternates, indicating an exact temporal correlation with the applied wave.

FIG. 11 shows another use of fluorescent membrane potentiometric probes in a bilayer. Here, the potentiometric dye is used to detect the presence of the bacterial pore-forming toxin alpha-hemolysin in the bilayer. There is a marked increase in fluorescence intensity when membrane protein is present in the bilayer.

### Droplet-On-Hydrated-Support Bilayers for Fluorescence

Droplet-on-hydrated-support bilayers laid down on thin hydrated supports can be fluorescently examined using total internal reflection fluorescence (TIRF) microscopy.

FIG. 13, which illustrates total internal reflection fluorescence microscopy on a droplet-on-hydrated-support bilayer, a supporting substrate comprised of a thin layer of agarose is formed on a glass coverslip. This thin substrate is rehydrated by filling a polymethyl methacrylate (PMMA) micro-channel device with aqueous agarose. The device wells are filled with a solution of lipid in oil. An aqueous droplet is placed on top of the hydrogel underneath the oil. A lipid bilayer forms at the interface between the two aqueous phases. The evanescent field propagates into the droplet-on-hydrated-support bilayer illuminating the lipid bilayer and fluorophores in the droplet.

TIRF techniques may also be used in combination with other analysis techniques, for example, in combination with acquiring electrical data. The combination of data may provide improved information on protein function.

### Further Examples of the Introduction of Cell Membrane Proteins During Bilayer Formation

To further demonstrate the feasibility of the method of the invention a number of representative ligand- and voltage-gated ion channels were studied. A range of different conditions; ranging from prokaryotic proteins, to primary tissue samples from mammals were studied. Ion channel activity was recorded in bulk as well as from single channels. Currents were recorded from a number of over-expressed eukaryotic proteins including the NMDA receptor, hERG and KATP. Endogenously expressed channels were also studied. Single-channel recordings of ion current from cells that are currently difficult to patch clamp due to their size or shape, including sickle cells and mitochondria, were obtained.

As described above the cell membrane fragments were introduced into the lipid bilayers on bilayer formation.

### Methods

### Cell Lines, Proteins, Preparation of Membranes, and Electrical Recording

KcsA was overexpressed in pQE60 (Qiagen, Germany) using a porin-free E. coli strain PC2889. Cells were disintegrated using a pressure-based cell homogenizer (TC5-612, Stansted Fluid Power, UK) and membranes isolated by centrifugation in water before freezing in liquid nitrogen. Sedimented membranes were resuspended in 150 mM KCl, 10 mM Hepes, pH 7.5. Membrane preparations were diluted 1:10⁶ (for all cases dilutions are reported as volume:volume unless stated otherwise) and 1:10⁸ for bulk and single channel recordings, respectively. Bulk and single channel recording was performed in 150 mM KCl, 10 mM Succinic acid, pH 4. Blocking experiments were performed using droplets containing 150 mM BaCl₂, 10 mM Hepes, pH 7.5 (+50 mV, n=5).

hERG.

Growth arrested HEK293 cells over-expressing the KCNH2 gene (Invitrogen, UK) stored at −80° C., were sonicated (Sonorex, Bandelin, Germany, 320 W, 4° C., 3 minutes). Membranes were then isolated by centrifugation, washed in buffer (350 mM KCl, 10 mM Hepes, pH 7.5) Finally membranes were diluted 1:10⁴ into the same buffer. Membranes were then incorporated into cooling low melting agarose (Sigma, total volume 10 μL, 1:10 sample:agarose, 1.5%, <60° C.) and deposited onto the coverslip prior to device assembly. Voltage-dependent current response was measured with and without the methanesulfonanilide drug E-4031 (1 mM E-4031, n=5, Invitrogen, UK), with +100 mV applied voltage For the recording of single channels, we dilution of the cell membrane preparation further (1:20).

NMDA Receptor.

Human NR1-1a/NR2A receptor subunits were expressed in a adherent mouse fibroblast 3T3 cell line. After mechanically detaching cells from culture flasks, cells were washed in isotonic buffer (3 times with 140 mM NaCl, 10 mM Hepes, pH 7.5), Cells were then mechanically disrupted by 30 s of ultrasound (Sonorex, Bandelin, Germany, 320 W) and four freeze-thaw cycles. After isolating the membranes by centrifugation and washing in the above buffer, the sample was diluted into the same buffer (1:10³). The agarose containing the membranes was then deposited onto the coverslip prior to device assembly. Membranes were then incorporated into cooling agarose (1.5%) before solidification with a total dilution of 1:5000 for bulk current recordings and 1:500000 for single channel levels, respectively. Addition of 10 μM glutamate and 10 μM glycine in 140 mM NaCl, 10 mM Hepes, pH 7.5, resulted in activation of the receptor after depolarization of the membrane (+50 mV applied voltage, n=5). Upon further dilution of the membrane preparation (1:100) single channels could be recorded (+50 mV applied voltage).

KATP.

For KATP channels, Kir6.2 and SUR1 (Sulfonylurea receptor) were co-expressed in Min6 cells. Frozen cells were subjected to three freeze-thaw cycles before they were sonicated for 1 minute (4° C., Sonorex, Bandelin, Germany, 320 W). After isolation of membranes by centrifugation, membranes were washed in isotonic buffer (140 mM KCl, 10 mM Hepes, pH 7.5), and diluted 1:5000. Membranes were incorporated into agarose by mixing (1:10 v:v) with cooling agarose (1.5%) and deposited onto the coverslip prior to device assembly. For single channel recordings the sample was diluted a further 10-fold. Electrical recordings were performed in 140 mM KCl, 10 mM Hepes, pH 7.5 in both droplet and agarose support. Bulk current block was obtained using glybenclamide (1 mM, +50 mV, n=5).

Lymphocytes.

SupT1 cells were grown as a suspension culture and washed four times in buffer (150 mM KCl, 10 mM Hepes, pH 7.5). Cells were then lysed by hypo-osmotic shock, and membranes isolated by centrifugation and washing in isotonic buffer (140 mM KCl, 10 mM Hepes, pH 7.5). Membranes were diluted 1:10⁴ and 1 μL of this solution was deposited on an agarose coated coverslip (1.5%). For bulk current blocking 4-aminopyridine a non-specific K+ channel blocker was used (5 mM, +50 mV). For specific blocking Kaliotoxin, a specific Kv-channel blocker, was used (50 μM, +50 mV).

Erythrocytes.

Following previously published protocol (Gibson et al, J Physiol: 511(Pt1), 225-234 (1998)), red blood cells were isolated from whole blood of Sickle Cell or healthy individuals by centrifugation and washing four times in PBS buffer (137 mM NaCl, 2.7 mM KCl, 10 mM sodium phosphate dibasic, 2 mM potassium phosphate monobasic, pH 7.4, containing 1% glucose), removing remaining blood components by aspiration. Cells were lysed by hypo-osmotic shock in low salt buffer (15 mM NaCl, 10 mM EDTA, 10 mM Hepes, pH 7). Membranes were then sedimented by centrifugation and washed in low salt buffer four times before resuspension into isotonic buffer (150 mM NaCl or KCl or CaCl₂, 10 mM Hepes, pH 7.4). For Gardos channel experiments, membranes were resuspended in isotonic buffer containing 150 mM KCl, 10 mM Hepes, pH 7. Droplets with or without Ca²⁺ (150 mM KCl, +/−5 mM CaCl₂, +/−5 mM KCl, 10 mM Hepes, pH 7, +50 mV, n=5) were used to observe Ca²⁺ dependent K⁺ flux. For electrical recordings of sickle cell and control membranes, membrane preparations were diluted 1:10⁵ into isotonic buffer (150 mM NaCl or KCl or CaCl₂, 10 mM Hepes, pH 7.4) and incorporated into agarose (1.5%, v/v 1:10 dilution) which was then deposited onto the coverslip prior to device assembly. Further dilution of 1:20 was needed for single channel recordings.

Mitochondria.

Following previously published protocol (Basoah et al FEB Lett: 579, 6511-6517 (2005)), pig liver cells were lysed on ice using a Teflon glass homogenizer driven by a motorised stirrer (Heidolph, Germany). Large debris, including intact cells, was removed by centrifugation at 1,100 g for 10 min at 4° C. Mitochondria were first pelleted by spinning at 11,000 g for 10 min at 4° C., and then washed once with 5 ml PBS, before spinning at 11,000 g for 5 min to produce the final mitochondrial pellet. Mitochondrial pellets were resuspended in ice cold oxygen consumption buffer (0.25M sucrose, 5 mM MOPS, 5 mM KH₂PO₄ and 5 mM MgCl₂, pH 7.4) and stored at −80° C. After thawing mitochondria were washed twice in isotonic potassium buffer (150 mM KCl, 10 mM Hepes, pH 7.5), the sonicated for 5 minutes (Sonorex, Bandelin, Germany, 320 W) and diluted 1:104 into buffer (150 mM KCl, 10 mM Hepes, pH 7.5). Membranes were then diluted 1:10 into cooling agarose (Sigma, total volume 10 μL, 1.5%) and deposited onto the coverslip prior to device assembly.

### Reconstitution and Assembly of a Droplet Bilayer Device.

By varying the dilution of the membrane fragments from 1:10⁴ to 1:10⁷ from a starting concentration of 5×10⁶ cells/mL, it is possible to control the number of channels in the bilayer. Channel incorporation can be achieved either by adding cell fragments to cooling but not solidified 1-1.5% agarose support in a 1:10 ratio (total volume 10 μL) or by pipetting the membranes on top of the hydrogel support. Microchannel devices were micro-fabricated from poly(methyl methacrylate) as published previously (Thompson et al, Nano Lett: 7, 3875-3878 (2007)). The device was placed on top of the slide before the agarose support layer is immersed in hexadecane-DPhPC solution (10 mM 1,2-diphytanoyl-sn-glycero-3-phosphocholine in anhydrous hexadecane, Sigma). Buffer containing droplets are first pipetted into a separate lipid-in-oil solution which allows the formation of a monolayer around the droplets. After a short period of equilibration in these lipid-in-oil solutions (2-15 minutes), a monolayer of lipid assembles at the interface between the aqueous phase and the hydrophobic lipid-in-oil solution. Droplets are then introduced onto prepared lipid-equilibrated agarose substrate by pipetting. Upon contact with the lipid monolayer on the surface of the hydrogel, a bilayer is formed. Droplet bilayers were imaged using an inverted microscope (Ti-Eclipse; Nikon Instruments).

Electrical Recording.

Bulk and single channel recordings were performed using our previously described method (Heron et al, J Am Chem Soc: 129, 16042-16047 (2007)). Using a micromanipulator, an electrode was inserted into the droplet (100 μm diameter, Ag/AgCl, Nashege, Japan. FIG. 1A). Current are measured between this electrode and a corresponding Ag/AgCl ground electrode in the agarose support. Currents were recorded using a patch clamp amplifier (Axopatch 200B; Axon Instruments). The signals were digitized using a NI-DAQ (National Instruments, UK). Electrical traces were filtered post-acquisition (100 Hz low pass Gaussian filter) and analyzed using WinEDR (University of Strathclyde, Institute of Pharmacy and Biomedical Sciences). Lipid bilayers were typically able to withstand voltages of 150 mV while retaining seals in excess of 100 GΩ. Electrical noise levels were typically of the order of ±01.0 pA rms (100 kHz recording bandwidth).

Bulk Current Blocking.

After reconstitution of the respective cell line bulk current of droplets with and without blocker were measured and mean values and standard deviation determined, n=5 both with and without blocker in all cases.

Electrical Recording of Ion Channels from Recombinant Over-Expressing Cells

Isolated membranes from a porin-free E. coli strain over-expressing the bacterial potassium channel KcsA were incorporated into bilayers according to the method of the invention. Membrane preparations were diluted into isotonic buffer before being incorporated into the agarose support. Addition of Ba²⁺ resulted in a 93% block in current (FIG. 21a). Upon further dilution of the membrane preparation single channels with low open probability and a conductance of 62+/−2.6 pS (FIG. 21a) were observed. Control experiments using E. coli cells without transformation showed no ionic current.

Eukaryotic Ion Channel Reconstitution into Droplet Bilayers

The steps required for reconstitution of eukaryotic ion channels into droplet bilayers are depicted in FIG. 20 wherein (1) Eukaryotic cells are ruptured; (2) Cell membranes are isolated and diluted; (3) Membranes are incorporated into the hydrogel; (4) A mixture of phospholipid in oil is then applied, and a monolayer self-assembles at the hydrogel-oil interface; (5) A nanolitre aqueous droplet is introduced, which also acquires a second lipid monolayer; (6) A bilayer forms when both monolayers are brought into contact. Spontaneous insertion of ion channels occurs during bilayer formation.

This method for the reconstitution eukaryotic ion channels can be adapted to a variety of samples and conditions. In the examples included herein cells were derived from tissue (FIG. 23b), blood (FIG. 23a, c, d) or cell culture (FIG. 21b-d, 22); from a suspension (FIG. 22) or adherent cell line (FIG. 21b-d). Cell disruption was achieved using ultrasound (FIG. 21), freeze-thaw, (FIG. 21), hypo-osmotic shock (FIG. 23a, c, d) or extrusion. Incorporation of the membranes into the hydrogel support was achieved by adding cell fragments during support creation, or by pipetting the membrane solution onto the hydrogel support. By varying the dilution of the membrane fragments, it was also possible to control the number of channels in the bilayer.

To test the generality of this method of the invention a range of representative eukaryotic ion channels were selected. Firstly, ionic flux across membranes derived from cells over-expressing the voltage-gated potassium channel Kv11.1 (gene: KCNH2) were measured. The Kv11.1 (or hERG) channel is crucial to repolarization during the cardiac action potential¹⁸. Growth arrested HEK293 cells over-expressing hERG were mechanically disrupted by sonication, and membranes isolated by centrifugation and washed. Finally, membranes were resuspended into buffer (350 mM KCl, 10 mM Hepes, pH 7.5) and diluted before incorporation into the agarose layer. A voltage-dependent bulk current response was observed and ion current was blocked (97% FIG. 21b) by the hERG blocker, E-4031¹⁹. Upon further dilution of the cell membrane preparation single channel traces were recorded with an average conductance of 8+/−0.21 pS similar to previous reported values (FIG. 21b).

The ligand-gated ionotropic N-methyl-D-aspartate (NMDA) receptor was also studied. Mouse fibroblast (ltk−) cells expressing recombinant human NMDA receptor were washed in isotonic buffer before mechanical disruption, isolation of the membranes, and dilution (140 mM NaCl, 10 mM Hepes, pH 7.5). Membranes were then incorporated into the agarose layer before bilayer formation. Addition of glutamate (10 μM) and glycine (10 μM) showed an 98% increase in ionic flux consistent with activation of the receptor (FIG. 21c). When the cell preparation was further diluted single channels were observed that exhibited slow opening and closing events typical of the NMDA receptor. Observed conductance levels with 47.3 pS (+/−3.2) and a sub-conductance level of 36.2 pS (+/−3.55) correspond to values previously reported in the literature.

Ion flux across membranes derived from Min6 cells over-expressing K_{ATP} were measured. These channels consist of four Kᵢᵣ 6.2 and four Sulfonylurea receptor subunits which assemble on the membrane to form an active complex. Bulk currents were blocked (91%; FIG. 21d) by glibenclamide (1 mM), a specific K_{ATP} channel blocker²⁵. Further dilution of the cell preparation revealed single channels (FIG. 21d) with a conductance of 50+/−3.1 pS similar to previous observations in oocytes.

Electrical Recording of Ion Channels from Primary Cells and Mitochondria

Single channel currents and the voltage dependence of bulk current from endogenously expressed voltage-gated potassium channels in primary lymphocytes (FIG. 22a) were recorded. Lymphocyte potassium channels are important for signaling during immune response and regulate membrane potential and calcium signaling of T cells. A suspension of SupT1 cells were lysed by hypo-osmotic shock, and membranes isolated by centrifugation and washed in isotonic buffer. Membranes were then diluted before being deposited onto an agarose-coated coverslip. Voltage-gated behaviour typical for Kᵥ1.3 was observed, with increased open probability at higher voltages (FIG. 22b). The identity of Kᵥ1.3 was confirmed using non-specific (5 mM 4-aminopyridine, 96% block, FIG. 22D) K⁺ and specific Kv-channel blockers (50 μM Kaliotoxin, 99% block, FIG. 22E). From this pharmacological profile and the current voltage behaviour (FIG. 22B) the channel was identified as Kᵥ1.3.

One advantage of the method of the invention is that it is possible to take measurements of ion currents from membranes that would be extremely difficult to access using conventional patch clamping. This was examined using both erythrocytes and mitochondrial membranes.

Due to their size, erythrocytes represent one of the smallest and most difficult cells accessible by patch clamp. After isolation of erythrocytes from whole blood, the cells were lysed by hypo-osmotic shock, and membranes are resuspended in isotonic buffer. By conducting experiments with or without Ca²⁺ (5 mM) a Ca²⁺ induced K⁺ flux was observed which increased seven fold in the presence of calcium, consistent with currents derived from the Gardos channel (FIG. 23a). The Gardos K⁺ ion channel is the most prevalent channel in membranes of erythrocytes.

Membranes of red blood cells were reconstituted from both healthy individuals and those suffering from sickle cell disease. Sickling is known to occur via a change in the cation permeability of erythrocyte cell membranes and leads to dehydration of the cell; however, the molecular basis of this initial step is still the subject of debate.

Erythrocyte membranes were incorporated into the hydrogel and studied in the method of the invention. Reconstituted membranes from healthy individuals showed very little or no conductance, membranes from sickle cells were highly conductive for Na⁺, K⁺ and Ca⁺ with an essentially Ohmic response (FIG. 23c). Upon further dilution of the sample preparation (1:20) single channel recordings were obtained from membranes derived from sickle cells which were absent in membranes from healthy individuals (FIG. 23c). Clearly this experiment alone cannot attribute this channel activity to a specific channel, however, it does serve to highlight the case with which ion channels present within erythrocyte membranes can be studied using this technique.

Organelles and cells smaller than erythrocytes are very difficult to investigate with conventional patch clamping. Since the method of the invention is not limited by the size of the studied cell, mitochondrial membranes isolated from porcine liver were reconstituted in bilayers according to the invention. After mechanical disruption of isolated mitochondria, the membranes were separated and washed in isotonic buffer, before reconstitution into a droplet bilayer. Both single channel and bulk currents were resolved using either potassium or calcium salts (FIG. 23b).

### Discussion and Applications

The data presented herein shows the successful reconstitution of membrane proteins from a wide variety of ion channel types and sample conditions. These experiments required no specialist equipment beyond a sensitive current amplifier, and membrane protein reconstitution was reliable and reproducible in all cases.

Using the method of the invention the preparation of eukaryotic membrane protein-containing DHBs is rapid, straightforward, and can be easily scaled to massively parallel measurement in a DHB array. It is possible to monitor ionic currents and fluorescence signals from individual bilayers present in a DHB array (˜10̂12 bilayers cm⁻²). DHB arrays containing eukaryotic membrane proteins would permit the response of different membrane proteins to electrical and chemical stimuli to be measured.

A key feature of the method of the invention is the incorporation of ion channels directly from cell membranes in a synthetic lipid bilayer without the need for detergent purification. Detergent-free reconstitution protocols have been reported since the 1970s, however, these approaches can be unreliable and require considerable sample preparation. The lack of recent work in this area is perhaps an indicator of the difficulties in achieving efficient protein incorporation using this method.

The relative ease with which both prokaryotic and eukaryotic channels are incorporated into the bilayer demonstrates that for many experiments the method of the invention is a useful alternative to patch clamping. The reconstitution of ion channels from erythrocytes and mitochondria also demonstrates that the method of the invention has application in the study of other small cells and cellular compartments that are currently inaccessible to all but the most skilled patch clampers. At present smaller objects can only be patched through nanoscopic control of the patch pipette, by the use of giant proteoliposomes or by studying atypically large variants of the membrane component. Using the method of the invention the recording of single channels can be achieved with relatively straightforward sample preparation and without the need for microscopic positioning of a patch pipette.

Ion channels remain an under-exploited target class, largely due to the lack of techniques capable of high-throughput interrogation of channel function. As multiple droplet bilayers are simple to produce using the method of to invention there in now potential a tool for high throughput screening of ion channels, using both over-expressed targets and primary cells.

For example, it may be possible to screen drug libraries for binding affinities against hundreds of cloned ion channels in parallel, with resolution greater than that of whole cell patch-clamp electrophysiology. This approach could yield information about potential unwanted side-effects during drug development.

Due to the ability to rapidly screen much larger numbers of membrane proteins than in current methods, safety trials might also be performed on clones of different isoforms and mutants of membrane proteins not often found in common drug testing on patients, i.e. where mutants are present in a small fraction of the population. As it is possible to reconstitute proteins from primary tissue samples, where only a few cells are required. Drugs could be screened for side-effects on patient tissue at the point of delivery.

The low quantities of material required for this reconstitution method (less than one cell per measurement), electrical control, and access to both sides of the bilayer are particular advantages of this technique. The ability to image the bilayer also opens up a wide range of optical measurements of membrane protein responses. As well as ion channels the method of the invention can be used to interrogate other classes of membrane protein including receptor kinases, G protein-coupled receptors and transporters.

### Drug Safety and Long QT Screening.

The QT interval of the electrocardiogram measures ventricular repolarization. This interval can be detrimentally lengthened by the interaction of certain drugs with ion channels. Prolongation of the QT interval can lead to tachyarrhythmia and torsade-de-pointes, and if this occurs in conjunction with cardiac disease or other heart illnesses it may lead to sudden death. Inhibition of the hERG (Kv11.1) ion channel is the most common cause of QT interval elongation.

Since the discovery that this delayed ventricular repolarisation can be induced by certain FDA-licensed drugs, several national bodies controlling drug licensing have made screening of new compounds against hERG a regulatory requirement. Screening of new compounds for detrimental changes in hERG ionic currents represents a major challenge for pharmaceutical companies. The need for a low-cost high-throughput screening assay for the hERG channel is now a necessity in the pharmaceutical industry.

Current in vitro screening methods involve voltage-clamp, fluorescence, or binding assays. Of such methods, only voltage-clamp current recordings currently provide the ‘gold standard’ in determining hERG inhibition.

Preliminary experiments have shown that prototype arrays of DHBs as large as several hundred bilayers can be created with a density of 1012 bilayers per cm². Single-channel or bulk electrical recordings from each individual bilayer may be recorded. The current response from each bilayer may be imaged using fluorescence microscopy.

FIG. 12 illustrates that reagents can be introduced into an aqueous droplet 609 by injection from a micro-pipette 619 or a micro-pipette with a multi bore capillary 621. This offers a simple way of introducing compounds into an existing droplet.

### Device for Producing an Array of Bilayers

FIG. 14 depicts a device which can be used to produce an array of bilayers. The device comprises a base 842 and a lid 841. The base 840 is filled with agarose in the lower channel, then filled with a lipid/oil solution and left to equilibrate so that a monolayer forms on the agarose substrate. Cell membrane fragments or whole cells may be pipetted onto the agarose for incorporation into the bilayer. The lid 841 is dipped in a bulk aqueous solution, and through hydrophilic interaction with the plastic used to make the lid, small droplets remain on the lid. The lid is then lowered into the base, such that the droplets remain in oil for a time to equilibrate, before the lid is then lowered further to bring the droplets into contact with the underlying agarose. An array of bilayers then forms spontaneously where the droplets contact the agarose surface. By using electrodes connected to each of the droplets through the lid, and with a common electrode in the agarose, each bilayer in the array is individually accessible for electrical measurements. The nature of the device allows individual bilayers 844 to be imaged from below with a microscope.

As shown in FIG. 15, an array of bilayers can be formed with a hexagonal PDMS stamp, used for experimental screens. 150 mm diameter wells were fabricated resulting in a bilayer density of 360 bilayers/cm².

1-52. (canceled)

53. A method of producing a bilayer of amphipathic molecules comprising:
(a) providing a hydrated support and a hydrophilic body immersed in a hydrophobic medium;
wherein a first monolayer of amphipathic molecules is formed on an interface between the hydrophobic medium and the hydrophilic body and a second monolayer of amphipathic molecules is formed on an interface between the hydrophobic medium and the hydrated support; and
(b) bringing the first monolayer into contact with the second monolayer to form a bilayer of amphipathic molecules,
wherein at least part of a cell membrane, comprising cell membrane constituents, is provided in or on the hydrated support and/or in the hydrophilic body such that constituents of the cell membrane incorporate into the bilayer during or after the bilayer formation.

54. The method of claim 53, wherein the amphipathic molecules are provided in the hydrophobic medium.

55. The method of claim 53, wherein the amphipathic molecules are provided in the hydrophilic body and on the hydrated support.

56. The method of claim 53, wherein the cell membrane is provided in the hydrated support and/or provided in the hydrophilic body after the bilayer has formed.

57. The method of claim 53, wherein the cell membrane is provided in and/or on the hydrated support and/or in the hydrophilic body before forming the bilayer.

58. The method of claim 53, wherein the cell membrane is incorporated at the same time as the formation of the bilayer.

59. The method of claim 53, wherein step (a) involves:
providing a hydrated support;
providing cell membrane, by either layering cell membrane on top of the hydrated support, or alternatively by providing the hydrated support in a form where cell membrane is mixed in the hydrated support; and then
immersing the support in a hydrophobic medium that contains amphipathic molecules, resulting in a monolayer of amphipathic molecules being formed on an interface between the hydrophobic medium and the hydrated support; and then
immersing a hydrophilic body in the hydrophobic medium, resulting in a monolayer of amphipathic molecules being formed on an interface between the hydrophobic medium and the hydrophilic body.

60. The method of claim 53, wherein the at least part of a cell membrane is provided as a whole cell membrane.

61. The method of claim 53, wherein the at least part of a cell membrane is provided as a fragment of a cell membrane.

62. The method of claim 53, wherein the at least part of a cell membrane is provided as a subcellular membrane preparation, or as a subcellular organelle intact or fragmented, or in the form of liposomes derived from cell membrane fragments or liposomes derived from purified components, or cell membrane fragments derived from the plasma membrane of a cell, or a sub-cellular compartment.

63. The method of claim 53, wherein the cell membrane is eukaryotic.

64. The method of claim 53, wherein the cell membrane is prokaryotic viral.

65. The method of claim 53, wherein the hydrophilic body comprises a droplet of aqueous solution.

66. The method of claim 53, wherein the amphipathic molecules are lipid molecules.

67. The method of claim 53, wherein the hydrated support comprises a solid or a semi-solid substrate.

68. The method of claim 53, wherein the hydrated support is hydrophilic.

69. The method of claim 53, wherein the hydrated support is porous.

70. The method of claim 53, wherein the hydrated support is non-porous.

71. The method of claim 53, wherein the amphipathic molecules are limpid molecules selected from the group comprising fatty acyls, glycerolipids, glycerophospholipids, sphingolipids, sterols, prenol lipids, polyketides, phospholipids and glycolipids.

72. The method of claim 71, wherein the lipid includes any limpids of the group comprising: monoolein; 1,2-dioleoyl-sn-glycero-3-phosphocholine (DOPC); 1,2-diphytanoyl-sn-glycero-3-phosphatidylcholine (DPhPC); palmitoyl oleoyl phosphatidylcholine (POPC); 1-palmitoyl-2-oleoyl-phosphatidylethanolamine (POPE); 1-palmitoyl-2-oleoyl-phosphatidylethanolamine; and 1-palmitoyl-2-oleoylphosphatidylglycerol (POPE/POPG) mixtures; or mixtures thereof.
